SEQUENCE LISTING (19 sequences)

SEQ ID NO: 1 - protein, 12 aa, synthetic construct
  SITE 1: note = Acetylated residue
  SITE 3: note = Amino isobutyric acid
  SITE 8: note = Amino isobutyric acid
  SITE 12: note = Amidated residue
ASXLRKLXKRLL

SEQ ID NO: 2 - protein, 12 aa, synthetic construct
  SITE 1: note = Picolinyl alanine
  SITE 3: note = Amino isobutyric acid
  SITE 8: note = Amino isobutyric acid
  SITE 12: note = Amidated residue
ASXLRKLXKRLL

SEQ ID NO: 3 - protein, 13 aa, synthetic construct
  SITE 1: note = Picolinyl alanine
  SITE 8: note = Amino isobutyric acid
  SITE 13: note = Amidated residue
  DISULFID 3..13: note = Intrachain disulfide bond
ASCLRKLXKRLLC

SEQ ID NO: 4 - protein, 12 aa, synthetic construct
  SITE 1: note = Acetylated residue
  SITE 5: note = Amino isobutyric acid
  SITE 10: note = Amino isobutyric acid
  SITE 12: note = Amidated residue
LLRKXLKRLXSA

SEQ ID NO: 5 - protein, 12 aa, synthetic construct
  SITE 1: note = Acetylated D-amino acid
  SITE 2: note = D-Amino acid
  SITE 3: note = D-Amino acid
  SITE 4: note = D-Amino acid
  SITE 5: note = D-Amino isobutyric acid
  SITE 6: note = D-Amino acid
  SITE 7: note = D-Amino acid
  SITE 8: note = D-Amino acid
  SITE 9: note = D-Amino acid
  SITE 10: note = D-Amino isobutyric acid
  SITE 11: note = D-Amino acid
  SITE 12: note = Amidated D-amino acid
LLRKXLKKLXSA

SEQ ID NO: 6 - protein, 12 aa, synthetic construct
  SITE 1: note = Acetylated D-amino acid
  SITE 2: note = D-Amino acid
  SITE 3: note = D-Amino isobutyric acid
  SITE 4: note = D-Amino acid
  SITE 5: note = D-Amino acid
  SITE 6: note = D-Amino acid
  SITE 7: note = D-Amino acid
  SITE 8: note = D-Amino isobutyric acid
  SITE 9: note = D-Amino acid
  SITE 10: note = D-Amino acid
  SITE 11: note = D-Amino acid
  SITE 12: note = Amidated D-amino acid
ASXLRKLXKRLL

SEQ ID NO: 7 - protein, 23 aa, synthetic construct
  SITE 1: note = Acetylated residue
  SITE 5: note = Amino isobutyric acid
  SITE 10: note = Amino isobutyric acid
  SITE 14: note = Amino isobutyric acid
  SITE 19: note = Amino isobutyric acid
  SITE 23: note = Amidated residue
LLRKXLRKLXSASXLRKLXKRLL

SEQ ID NO: 8 - protein, 17 aa, synthetic construct
  SITE 1: note = Acetylated residue
  SITE 8: note = Amino isobutyric acid
  SITE 13: note = Amino isobutyric acid
  SITE 17: note = Amidated residue
LRVRCASXLRKLXKRLL

SEQ ID NO: 9 - protein, 17 aa, synthetic construct
  SITE 1: note = Acetylated residue
  SITE 8: note = Amino isobutyric acid
  SITE 13: note = Amino isobutyric acid
  SITE 17: note = Amidated residue
LRVRLASXLKKLXKRLL

SEQ ID NO: 10 - protein, 17 aa, synthetic construct
  SITE 1: note = Acetylated residue
  SITE 8: note = Amino isobutyric acid
  SITE 13: note = Amino isobutyric acid
  SITE 17: note = Amidated residue
LRVRLASXLRKLXKRLL

SEQ ID NO: 11 - protein, 17 aa, synthetic construct
  SITE 1: note = Acetylated D-Amino acid
  SITE 2: note = D-Amino acid
  SITE 3: note = D-Amino acid
  SITE 4: note = D-Amino acid
  SITE 5: note = D-Amino isobutyric acid
  SITE 6: note = D-Amino acid
  SITE 7: note = D-Amino acid
  SITE 8: note = D-Amino acid
  SITE 9: note = D-Amino acid
  SITE 10: note = D-Amino isobutyric acid
  SITE 11: note = D-Amino acid
  SITE 12: note = D-Amino acid
  SITE 13: note = D-Amino acid
  SITE 14: note = D-Amino acid
  SITE 15: note = D-Amino acid
  SITE 16: note = D-Amino acid
  SITE 17: note = Amidated D-amino acid
LLRKXLKRLXSALRVRL

SEQ ID NO: 12 - protein, 29 aa, synthetic construct
  SITE 1: note = Acetylated residue
  SITE 20: note = Amino isobutyric acid
  SITE 25: note = Amino isobutyric acid
  SITE 29: note = Amidated residue
LRVRLASHLRKLRKRLLASXLRKLXKRLL

SEQ ID NO: 13 - protein, 12 aa, synthetic construct
  SITE 1: note = Alanine linked to 8 straight-chain carbon atom octanyl group
  SITE 3: note = Amino isobutyric acid
  SITE 8: note = Amino isobutyric acid
  SITE 12: note = Amidated residue
ASXLRKLXKRLL

SEQ ID NO: 14 - protein, 13 aa, synthetic construct
  SITE 1: note = Acetylated lysine linked to 8 straight-chain carbon atom octanyl group
  SITE 4: note = Amino isobutyric acid
  SITE 9: note = Amino isobutyric acid
  SITE 13: note = Amidated residue
KASXLRKLXKRLL

SEQ ID NO: 15 - protein, 13 aa, synthetic construct
  SITE 1: note = Acetylated picolinyl lysine
  SITE 4: note = Amino isobutyric acid
  SITE 9: note = Amino isobutyric acid
  SITE 13: note = Amidated residue
KASXLRKLXKRLL

SEQ ID NO: 16 - protein, 18 aa, synthetic construct
  SITE 1: note = Acetylated residue
  SITE 18: note = Amidated residue
LRVRLASHLRKLRKRLLR

SEQ ID NO: 17 - protein, 18 aa, synthetic construct
  SITE 1: note = Acetylated residue
  SITE 18: note = Amidated residue
LRKLRKRLLLRKLRKRLL

SEQ ID NO: 18 - protein, 30 aa, synthetic construct
  SITE 1: note = Acetylated residue
  SITE 30: note = Amidated residue
LRVRLASHLRKLRKRLLRDADDLQKRLAVY

SEQ ID NO: 19 - protein, 17 aa, synthetic construct
  SITE 1: note = Picolinyl D-lysine
  SITE 2: note = D-Amino acid
  SITE 3: note = D-Amino acid
  SITE 4: note = D-Amino acid
  SITE 5: note = D-Amino isobutyric acid
  SITE 6: note = D-Amino acid
  SITE 7: note = D-Amino acid
  SITE 8: note = D-Amino acid
  SITE 9: note = D-Amino acid
  SITE 10: note = D-Amino isobutyric acid
  SITE 11: note = D-Amino acid
  SITE 12: note = D-Amino acid
  SITE 13: note = D-Amino acid
  SITE 14: note = D-Amino acid
  SITE 15: note = D-Amino acid
  SITE 16: note = D-Amino acid
  SITE 17: note = Amidated D-amino acid
LLRKXLKRLXSALRVRL

(19) United States
(12) Patent Application Publication
(10) Pub. No.: US 2026/0007717 A1
(43) Pub. Date: Jan. 8, 2026

(54) ANTIMICROBIAL PEPTIDE COMPOUNDS AND METHODS OF USE

(71) Applicant: Regennova, Inc., Durham, NC (US)

(72) Inventors: Fengqiao Li, Chapel Hill, NC (US); Michael Peter Vitek, Efland, NC (US)

(21) Appl. No.: 18/992,791

(22) PCT Filed: Jul. 13, 2023
(86) PCT No.: PCT/US2023/070144

§ 371 Date: Jan. 9, 2025

Related U.S. Application Data
(60) Provisional application No. 63/368,505, filed on Jul. 15, 2022.

(51) Int. Cl. A61K 38/10; A61K 38/12; A61K 38/16; A61K 45/06; A61P 31/04

(52) U.S. Cl. CPC A61K 38/10; A61K 38/12; A61K 38/16; A61K 45/06; A61P 31/04

(57) ABSTRACT

Antimicrobial peptide compounds, pharmaceutical compositions, and methods for their use in inhibiting microbial growth are provided. The methods provided include methods for inhibiting pan drug resistant Acinetobacter baumannii by administering the peptide compounds to a subject. A method is provided for inhibiting pan drug resistant Acinetobacter baumannii with a synergistic combination of peptide compound Acetyl-AS-aib-LRKL-aib-KRLL-amide (SEQ ID NO: 1) and polymyxin B. In other methods, peptide compounds are provided for inhibiting P. gingivalis which is the keystone bacteria of periodontal disease, the 6^{th} most common infectious disease worldwide.

### CROSS-REFERENCE TO RELATED APPLICATIONS

This application claims priority to U.S. Provisional Patent Application No. 63/368,505, filed on Jul. 15, 2022, which is incorporated by reference herein in its entirety.

### FIELD OF INVENTION

The present invention relates to antimicrobial peptide compounds and specifically to their use in the treatment of microbial infection including for pandrug-resistant bacteria.

### BACKGROUND

The emergence of resistance to multiple antimicrobial agents in pathogenic bacteria has become a significant public health problem. Standardized definitions of the degree of resistance have been created for pathogenic bacteria using documents and breakpoints from the Clinical Laboratory Standards Institute (CLSI), the European Committee on Antimicrobial Susceptibility Testing (EUCAST) and the United States Food and Drug Administration (FDA). Multidrug resistant (MDR) was defined as acquired non-susceptibility to at least one agent in three or more antimicrobial categories, extensively drug-resistant (XDR) was defined as non-susceptibility to at least one agent in all but two or fewer antimicrobial categories (i.e. bacterial isolates remain susceptible to only one or two categories) and pandrug-resistant (PDR) was defined as non-susceptibility to all agents in all antimicrobial categories.

One particularly problematic bacterium is Acinetobacter baumannii. Acinetobacter baumannii is a Gram-negative opportunistic pathogen, causing serious nosocomial infections among immunocompromised patients. A striking characteristic of this bacterium is its capability of developing antimicrobial resistance rapidly and surviving on the dry surfaces of medical facilities for weeks. As early as 2009, carbapenem-resistant A. baumannii was listed among the six high threat pathogens, also known as “ESKAPE” pathogens by the Infectious Diseases Society of America, calling for global cooperation in response to this threat. Especially, the emergence of chromosomally encoded and plasmid-mediated polymyxin resistance in A. baumannii breaks through the last resort of defense. At present, the prevalence of A. baumannii infection is considered as a major and complex issue of global health.

In addition to multi-, extensively-, and pan-drug-resistant bacteria, P. gingivalis is the keystone bacteria of periodontal disease (PD), the 6th most common infectious disease worldwide. In the United States, half of all adults over the age of 30 and 70% of those over age 65 suffer some degree of P. gingivalis infection associated with gum disease. Older men and African-Americans are more at risk to develop periodontal disease. In a study of 6800 patients followed for up to 26 years, gingivitis followed by more serious periodontal disease was linked to an increased risk of dementia including Alzheimer's dementia. Tooth loss, a frequent outcome of PD, also increases the risk of dementia. P. gingivalis products including lipopolysaccharide (LPS), cysteine proteases known as gingipains, and 16S rRNA have all been found in human AD brains. Since P. gingivalis can be found in the mouth, the blood, and is known to invade non-oral tissues including the brain where it can induce a pro-inflammatory state that has also been linked to AD, then strategies to reduce P. gingivalis numbers and activities are already beginning to show positive effects on outcomes associated with AD.

Thus, there is an urgent and immediate need for new antimicrobials with activity against drug resistant bacteria including pan-drug-resistant bacteria and against P. gingivalis. The present disclosure provides such antimicrobials.

### SUMMARY

In one aspect of the present invention, a series of peptide compounds are provided that have antimicrobial activity. The peptide compounds of the present invention have a structure selected from:
(COG1410) acetyl-AS-aib-LRKL-aib-KRLL-amide (SEQ ID NO: 1);
Picolinyl-AS-aib-LRKL-aib-KRLL-amide (SEQ ID NO: 2);
Picolinyl-AS-C-LRKL-aib-KRLL-C-amide (SEQ ID NO: 3); wherein there is a
disulfide link between the two cysteine residues;
Acetyl-LLRK-aib-LKRL-aib-SA-CONH2 (SEQ ID NO: 4);
Acetyl-llrk-Aib-lkkl-Aib-sa-amide (SEQ ID NO: 5), wherein all the amino acid
residues arc D-amino acids;
Acetyl-as-aib-lrkl-aib-krll-amide (SEQ ID NO: 6), wherein all the amino acid residues
are D-amino acids;
Acetyl-LLRK-aib-LRKL-aib-SAS-aib-LRKL-aib-KRLL-CONH2 (SEQ ID NO: 7);
Acetyl-LRVRCAS-aib-LRKL-aib-KRLL-CONH2 (SEQ ID NO: 8);
Acetyl-LRVRLAS-aib-LKKL-aib-KRLL-Amide (SEQ ID NO: 9);
Acetyl-LRVRLAS-aib-LRKL-aib-KRLL-Amide (SEQ ID NO: 10);
Acetyl-llrk-aib-lkrl-aib-salrvrl-amide (SEQ ID NO: 11), wherein all the amino acid
residues are D-amino acids;
Acetyl-LRVRLASHLRKLRKRLLAS-aib-LRKL-aib-KRLL-CONH2 (SEQ ID NO: 12);
C8-AS-aib-LRKL-aib-KRLL-amide (SEQ ID NO: 13);
Acetyl-K(C8)-AS-aib-LRKL-aib-KRLL-amide (SEQ ID NO: 14);
Acetyl-K(Picolinyl)-AS-aib-LRKL-aib-KRLL-amide (SEQ ID NO: 15);
Acetyl-LRVRLASHLRKLRKRLLR-amide (SEQ ID NO: 16);
Acetyl-LRKLRKRLLLRKLRKRLL-amide (SEQ ID NO: 17);
Acetyl-LRVRLASHLRKLRKRLLRDADDLQKRLAVY-amide (SEQ ID NO: 18); and
Picolinyl-llrk-aib-lkrl-aib-salrvrl-amine (SEQ ID NO: 19), wherein all amino acid
residues are D-amino acids,
wherein aib is amino isobutyric acid.

In another embodiment, a peptide compound of the invention, or a pharmaceutically acceptable salt or solvate thereof, is provided for use in a method of inhibiting microbial growth in a subject.

In other aspects, a pharmaceutical composition comprising a peptide compound of the invention is provided for use in a method of inhibiting microbial growth in a subject.

The pharmaceutical composition may comprise the peptide compound SEQ ID NO: 1, SEQ ID NO: 2, SEQ ID NO: 3, SEQ ID NO: 13, SEQ ID NO: 14, or SEQ ID NO: 15. The pharmaceutical composition may comprise the peptide compound SEQ ID NO: 1, SEQ ID NO: 2, or SEQ ID NO: 3. In some instances, the pharmaceutical composition comprises the peptide compound SEQ ID NO: 1.

In other embodiments, the pharmaceutical composition comprises one or more other antimicrobial compounds. The one or more antimicrobial compounds may include polymixin B.

In one aspect, a method is provided for inhibiting microbial growth, comprising: contacting a microbe with an effective amount of a peptide compound of the present invention to inhibit the microbial growth. In certain embodiments of this method, the peptide compound can comprise SEQ ID NO: 1, SEQ ID NO: 2, SEQ ID NO: 3, SEQ ID NO: 13, SEQ ID NO: 14, or SEQ ID NO: 15. In another instance, the peptide compound can comprise SEQ ID NO: 1, SEQ ID NO: 2, or SEQ ID NO: 3. In some instances, the peptide compound comprises SEQ ID NO: 1.

In the method comprising contacting a microbe with an effective amount of a peptide compound of the present invention, the microbe can comprise a bacterium that is one or more of multidrug-resistant (MDR), extensively drug-resistant (XDR), or pandrug-resistant (PDR). In some cases, the microbe comprises Porphoryomas gingivalis. The microbe may comprise a Gram-negative bacterial pathogen or a Gram-positive pathogen. The Gram-negative bacterial pathogen may include one or a combination of Acinetobacter baumannii, P. gingivalis, or E. coli and the Gram-positive pathogen may include one or a combination of S. aureus or L. salivarius.

In some embodiments of the method comprising contacting a microbe with an effective amount of a peptide compound of the present invention, the peptide compound is contacted with the microbe in combination with one or more other antimicrobial compounds. The one or more antimicrobial compounds can include polymyxin B. The microbial growth inhibited in the method can include microbial growth due to pandrug-resistant Acinetobacter baumannii or multidrug-resistant S. aureus. The peptide can include SEQ ID NO: 1, SEQ ID NO: 2, or SEQ ID NO: 3, and the microbe being inhibited can include pandrug-resistant Acinetobacter baumannii. In other cases, the peptide includes SEQ ID NO: 1, SEQ ID NO: 2, SEQ ID NO: 3, SEQ ID NO: 13, SEQ ID NO: 14, or SEQ ID NO: 15, and the microbe being inhibited includes pandrug-resistant Acinetobacter baumannii. In one instance, the peptide includes SEQ ID NO: 1, and the microbe being inhibited includes pandrug-resistant Acinetobacter baumannii.

In another embodiment of the invention, a method is provided for treating a subject having a microbial infection, the method comprising administering to a subject a therapeutically effective amount of a pharmaceutical composition comprising a peptide compound of the invention, or a pharmaceutically acceptable salt or solvate thereof, to inhibit microbial growth in the subject. In the method, the pharmaceutical composition may comprise one or more other antimicrobial compounds. The one or more antimicrobial compounds may include polymixin B.

In the method of treating a subject having a microbial infection, the pharmaceutical composition may comprise the peptide compound SEQ ID NO: 1, SEQ ID NO: 2, SEQ ID NO: 3, SEQ ID NO: 13, SEQ ID NO: 14, or SEQ ID NO: 15. In other instances, the pharmaceutical composition may comprise the peptide compound SEQ ID NO: 1, SEQ ID NO: 2, or SEQ ID NO: 3. In one instance, the pharmaceutical composition comprises the peptide compound SEQ ID NO: 1.

In the method of treating a subject having a microbial infection, the microbial growth can be due to a bacterium comprising one or more of multidrug-resistant (MDR), extensively drug-resistant (XDR), or pandrug-resistant (PDR). In other cases, the microbial growth is caused by an oral infection with a bacterium comprising Porphoryomas gingivalis. The microbial growth may be caused by a Gram-negative bacterial pathogen or a Gram-positive pathogen. The Gram-negative bacterial pathogen bacterial pathogen may comprise one or a combination of Acinetobacter baumannii, P. gingivalis, or E. coli and the Gram-positive pathogen may be S. aureus or L. salivarius

In one embodiment of the invention, an antimicrobial composition is provided, comprising: a therapeutically effective amount of a peptide compound, or a pharmaceutically acceptable salt or solvate thereof, comprising SEQ ID NO: 1, SEQ ID NO: 2, SEQ ID NO: 3, SEQ ID NO: 13, SEQ ID NO: 14, or SEQ ID NO: 15; and a therapeutically effective amount of polymyxin B. In some instances, the antimicrobial composition can comprise SEQ ID NO: 1, SEQ ID NO: 2, or SEQ ID NO: 3. In one aspect, the antimicrobial composition comprises SEQ ID NO: 1.

In another embodiment, the antimicrobial composition is provided for use in a method of inhibiting microbial growth in a subject.

In other aspects, a pharmaceutical composition comprising the antimicrobial composition of the invention is provided for use in a method of inhibiting microbial growth in a subject.

Administering the pharmaceutical compositions of the present invention to a subject can include a single administration or multiple administrations of the pharmaceutical composition. The pharmaceutical composition comprising the peptide compound can be administered topically, enterally, systemically, or parenterally. In some instances, the pharmaceutical composition can be administered in a formulation of a mouth wash or a toothpaste. In some cases when the microbial growth is caused by an oral infection with a bacterium that includes Porphoryomas gingivalis, the pharmaceutical composition comprising the peptide compound can be administered in a formulation of a mouth wash or a toothpaste.

The pharmaceutical composition may include one or more pharmaceutically acceptable excipients. The one or more excipients may include, but is not limited to, disintegrants, diluents, binders, solvents, co-solvents, lubricants, pH adjusting agents, buffering agents, preservatives, dispersing agents, suspending agents, ointment bases, emulsifiers, emollients, penetration agents, surfactants, propellants, flavoring agents, sweetening agents, or drug release modifiers. The pharmaceutical composition may include a solvent such as one or both of physiological saline and glucose solution.

The pharmaceutical compositions of the invention may be formulated as a freeze-dried powder or as a liquid suitable for administration by injection, to cause slow release of the peptide compound, or as a spray. In other embodiments, the pharmaceutical compositions of the invention may be formulated as a mouth wash or as a toothpaste.

### BRIEF DESCRIPTION OF THE DRAWINGS

The features and advantages of the present invention will be more clearly understood from the following description taken in conjunction with the accompanying drawings. The accompanying Figures are provided by way of illustration and not by way of limitation. The foregoing aspects and other features of the disclosure are explained in the following description, taken in connection with the accompanying example figures (also “FIG.”) relating to one or more embodiments.

FIG. 1A is a graph illustrating the antimicrobial activity of COG1410 (SEQ ID NO: 1) and specifically the in vitro killing kinetics of COG1410 and polymyxin B (PMB) against PDR-A. baumannii YQ4 strain in PBS at 1×MIC and 5×MIC, respectively.

FIG. 1B is a graph illustrating the antimicrobial activity of COG1410 (SEQ ID NO: 1) and specifically the bactericidal efficacy of COG1410 in PBS with or without 50% human plasma. CFU were counted after incubation at 37° C. for 2 h.

FIG. 1C is a graph illustrating the antimicrobial activity of COG1410 (SEQ ID NO: 1) and specifically the bactericidal efficacy of COG1410 in different conditions was determined. Each experiment in FIGS. 1A-1C was done in triplicate and the values represented means±SD.

FIG. 2A is a graph illustrating that COG1410 (SEQ ID NO: 1) exhibits biofilm inhibition and eradication activities against PDR-A. baumannii and specifically prevention of biofilm formation by COG1410. Results were expressed as the biofilm mass measured using crystal violet staining (OD₆₀₀).

FIG. 2B is a graph illustrating that COG1410 (SEQ ID NO: 1) exhibits biofilm inhibition and eradication activities against PDR-A. baumannii and specifically eradication of established biofilm. Data were represented mean±SD of 8 replicates from three independent experiments. The statistical significance between each treatment and control were analyzed by Student's t test (unpaired), *p<0.5, *** p<0.001.

FIG. 3A is images illustrating that COG1410 (SEQ ID NO: 1) treatment permeabilized the cell membrane of PDR-A. baumannii. as seen with SEM observation of A. baumannii YQ4 exposed to 1×MIC COG1410 or 1×MIC polymyxin B. The cells in PBS served as a negative control.

FIG. 3B is images illustrating that COG1410 (SEQ ID NO: 1) treatment permeabilized the cell membrane of PDR-A. baumannii. as seen with TEM observation of A. baumannii YQ4 exposed to 1×MIC COG1410 or 1×MIC polymyxin B. The cells in PBS served as a negative control.

FIG. 3C is a graph illustrating that COG1410 (SEQ ID NO: 1) treatment permeabilized the cell membrane of PDR-A. baumannii and specifically the effect of 1×MIC COG1410 on ATP release (i.e., ATP leak) from A. baumannii YQ4. 1×MIC polymyxin B and 1×MIC tigecycline (TGC, 32 μg/ml) were used as positive and negative controls for ATP leakage, respectively. Untreated cells were negative controls as well.

FIG. 3D is a graph illustrating that COG1410 (SEQ ID NO: 1) treatment permeabilized the cell membrane of PDR-A. baumannii and specifically showing measurement of ROS level by DCFH-DA probe in the presence or absence of COG1410 (16 μg/ml). Rosup is the positive control. The statistical significance between each treatment and control was analyzed by One-way ANOVA method with post-test for multiple comparisons, * p<0.05, ** p<0.01.

FIG. 4 shows images illustrating that FITC-COG1410 entered the cytoplasms of A. baumannii, E. faecium, K. pneumoniae and S. aureus. The bacteria were treated with FITC-labeled-COG1410 and counter-stained with FM4-64 dye and observed by CLSM. The green fluorescence (i.e., shaded shapes) indicates the localization of FITC-COG1410 in the cells. Red fluorescence (i.e., open shapes) indicated the cytoplasmic membrane. Scale bar: 4 μm.

FIG. 5A is the first half of a graph showing treatment of COG1410 (SEQ ID NO: 1)-enriched genes involved in oxidation-reduction process. The whole transcriptome analysis of A. baumannii treated or untreated with COG1410 was performed by RNA-seq. The differentially expressed genes (DEGs) were analyzed by using the edgeR (v3.16.5). Gene ontology (GO) enrichment analysis of DEGs was implemented by clusterProfiler (v3.4.4). GO terms with FDR≤0.05 were considered significantly enriched by DEGs.

FIG. 5B is the second half of the graph described in FIG. 5A.

FIG. 6A is a graph illustrating that COG1410 (SEQ ID NO: 1) exhibits low hemolytic activity and specifically the hemolytic activity was determined by measuring the release of hemoglobin of human erythrocytes at 414 nm, which were exposed to different concentrations of COG1410. PBS and Triton X-100 (0.1%) were used as negative and positive controls, respectively.

FIG. 6B is a graph illustrating that COG1410 (SEQ ID NO: 1) exhibits medium cytotoxicity and specifically the cytotoxicity of COG1410 was evaluated by measuring the cell viability of normal human hepatic L02 cell treated with the increasing concentration of peptide using a CCK8 assay. Experiments were conducted in triplicate. Data indicated means means±SD.

FIG. 7A is a graph showing COG1410 (SEQ ID NO: 1) exhibited strong synergistic interaction with polymyxin B and specifically modification of LPS did not change the antimicrobial activity of COG1410 against A. baumannii. MIC was determined against A. baumannii wild-type strain ATCC19606 and the corresponding LPS-defective mutants with pmrAP102R and pmrAP102RmiaAI221V mutation in LB broth.

FIG. 7B is a graph showing COG1410 (SEQ ID NO: 1) exhibited strong synergistic interaction with polymyxin B and specifically the combination of 2 μg/ml COG1410 and 1 μg/ml polymyxin B (i.e., 2 COG1410+1 PBM) could completely inhibit bacterial growth within 20 h in the LB broth. The growth curve was measured in duplicate, with eight wells for each treatment in a 96-well plate each time. The representative one was displayed.

FIG. 7C is a graph showing COG1410 (SEQ ID NO: 1) exhibited strong synergistic interaction with polymyxin B and specifically the combination of COG1410 and polymyxin B (i.e., 2 COG1410+1 PBM) significantly reduced the CFU of A. baumannii YQ4 in PBS. Experiments were conducted in triplicate. Data indicated mean±SD values.

FIG. 8 is a graph illustrating that the combined therapy of COG1410 (SEQ ID NO: 1) and polymyxin B rescued infected nematodes. Specifically, C. elegans were pre-infected by A. baumannii YQ4 and transferred to a NGM plate supplemented with 16 μg/ml COG1410 or 2 μg/ml COG1410 and 1 μg/ml polymyxin B. The dead nematodes were counted every day for 2 weeks. The survival curve was analyzed by Kaplan-Meier method and the statistical significance was analyzed by Log-rank test. **, p<0.01.

### DETAILED DESCRIPTION

To promote an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Unless otherwise indicated, technical and scientific terms used herein have the same meaning as commonly understood by those of ordinary skill in the art to which this disclosure belongs.

The use herein of the terms “including,” “comprising,” or “having,” and variations thereof, is meant to encompass the elements listed thereafter and equivalents thereof as well as additional elements. As used herein, “and/or” refers to and encompasses all possible combinations of one or more of the associated listed items, as well as the lack of combinations when interpreted in the alternative (“or”).

Furthermore, the indefinite articles “a” and “an” preceding an element or component of the invention are not intended to limit the number of occurrences of the element or component. Therefore, “a” and “an” should be understood to include one or at least one, and unless the number is explicitly singular, the singular form of the element or component also includes the plural form.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated should be considered as expressly stated in this disclosure.

For the purposes of the specification, drawings, and claims, the terms “protein”, “polypeptide”, and “peptide” are herein used interchangeably.

The term “treatment” as used herein means administration of a composition of the present invention to an individual suffering from a microbial infection that results in partial or complete remission of the symptoms or prevents aggravation of the symptoms of the microbial infection after treatment. Therefore, treatment includes cure. As used herein, “efficacy” represents the effect caused by the treatment, which changes, generally changes, alleviates or ameliorates symptoms or characteristics of a microbial infection, or that cures a microbial infection. “Treatment” may also mean prolonging survival as compared to expected survival if not receiving treatment. “Treatment” refers to both therapeutic treatment and prophylactic or preventative measures in certain embodiments. By treatment is meant inhibiting or reducing an increase in pathology or symptoms when compared to the absence of treatment and is not necessarily meant to imply complete cessation of the microbial infection.

As used herein, the term “therapeutically effective amount” refers to the use of or the method of administering an amount of a composition of the invention that will achieve the desired therapeutic efficacy after being administered.

As used herein, the term “subject” includes a human patient and a non-human (animal) patient. The term “non-human animal” includes vertebrates, for example, mammals, such as non-human primates, sheep, cows, dogs, cats, and rodents such as mice and rats.

The inventors have discovered that a series of peptide compounds have excellent antimicrobial effects. The peptide compounds are provided as antimicrobials and in some embodiments are provided in compositions for administration to subjects for the treatment of a microbial infection. The peptide compounds of the invention are provided below:
(COG1410) acetyl-AS-aib-LRKL-aib-KRLL-amide (SEQ ID NO: 1);
Picolinyl-AS-aib-LRKL-aib-KRLL-amide (SEQ ID NO: 2);
Picolinyl-AS-C-LRKL-aib-KRLL-C-amide (SEQ ID NO: 3); wherein there is a disulfide link between the two cysteine residues;
Acetyl-LLRK-aib-LKRL-aib-SA-CONH2 (SEQ ID NO: 4);
Acetyl-llrk-Aib-lkkl-Aib-sa-amide (SEQ ID NO: 5), wherein all the amino acid residues are D-amino acids;
Acetyl-as-aib-lrkl-aib-krll-amide (SEQ ID NO: 6), wherein all the amino acid residues are D-amino acids;
Acetyl-LLRK-aib-LRKL-aib-SAS-aib-LRKL-aib-KRLL-CONH2 (SEQ ID NO: 7);
Acetyl-LRVRCAS-aib-LRKL-aib-KRLL-CONH2 (SEQ ID NO: 8);
Acetyl-LRVRLAS-aib-LKKL-aib-KRLL-Amide (SEQ ID NO: 9);
Acetyl-LRVRLAS-aib-LRKL-aib-KRLL-Amide (SEQ ID NO: 10);
Acetyl-llrk-aib-lkrl-aib-salrvrl-amide (SEQ ID NO: 11), wherein all the amino acid residues are D-amino acids;
Acetyl-LRVRLASHLRKLRKRLLAS-aib-LRKL-aib-KRLL-CONH2 (SEQ ID NO: 12);
C8-AS-aib-LRKL-aib-KRLL-amide (SEQ ID NO: 13);
Acetyl-K(C8)-AS-aib-LRKL-aib-KRLL-amide (SEQ ID NO: 14);
Acetyl-K(Picolinyl)-AS-aib-LRKL-aib-KRLL-amide (SEQ ID NO: 15);
Acetyl-LRVRLASHLRKLRKRLLR-amide (SEQ ID NO: 16);
Acetyl-LRKLRKRLLLRKLRKRLL-amide (SEQ ID NO: 17);
Acetyl-LRVRLASHLRKLRKRLLRDADDLQKRLAVY-amide (SEQ ID NO: 18); and
Picolinyl-llrk-aib-lkrl-aib-salrvrl-amine (SEQ ID NO: 19), wherein all the amino acid residues are D-amino acids.

The term “COG1410” is used herein interchangeably with the term “SEQ ID NO: 1”.

In the above peptide compounds of the invention, L, R, K, S, H, A, C, V, D, Q, and Y are the one-letter abbreviations of amino acids, respectively representing leucine, arginine, lysine, serine, histidine, alanine, cysteine, valine, aspartate, glutamine, and tyrosine. Those skilled in the art understand that in the peptide chain structure, the above-mentioned amino acids are in the form of amino acid residues. In the present invention, unless otherwise specified, the amino acids included in the peptide chain structure refer to amino acid residues.

Unless otherwise specified, two adjacent amino acids are connected by an amide bond (—CO—NH—), also known as a peptide bond. For example, -AS— means that the alanine residue is connected to the serine residue, and the two are connected by an amide bond, which in this case, because A and S and amino acids, is also known as a peptide bond.

In the above peptide compounds, L, R, K, S, H, A, C, V, D, Q, and Y may represent L-amino acids unless it is stated otherwise that the amino acid residues are D-amino acids.

In the peptide compounds of the invention, “Aib” is amino isobutyric acid.

In the peptide compound, SEQ ID NO: 3, there is a disulfide bond between the side chains of the two cysteine residues (i.e., —SS— or a disulfide group or a disulfide bridge).

In peptide compounds SEQ ID NOs: 1, 4-12, and 16-18, the amino terminal amino acid is bonded to acetyl (i.e., acetyl-CONH—). For the peptide compounds that start with acetyl-AS, i.e., SEQ ID NOs: 1 and 6, an exemplary schematic of the structure of the amino terminus is shown below:

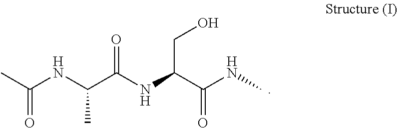

Structure (I) shows acetyl-Alanine-Serine with an acetyl group at the amino terminus.

For peptide compounds that begin with acetyl-LLRK, i.e., SEQ ID NOs: 4, 5, 7, and 11, the non-acetylated amino terminus is shown in structure (II) below:

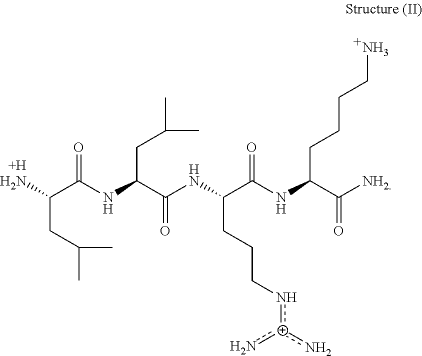

The acetyl-LLRK structure of SEQ ID NOs: 4, 5, 7, and 11 is shown below in structure (III):

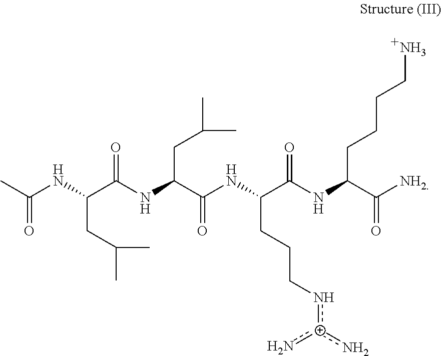

For the peptide compounds that begin with acetyl-LRVR, i.e., SEQ ID NOs: 8-10, 12, 16, and 18, the non-acetylated structure is shown below in structure (IV):

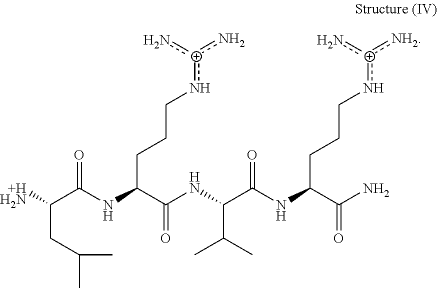

The acetyl-LRVR structure of SEQ ID NOs: 8-10, 12, 16, and 18 is shown below in structure (V):

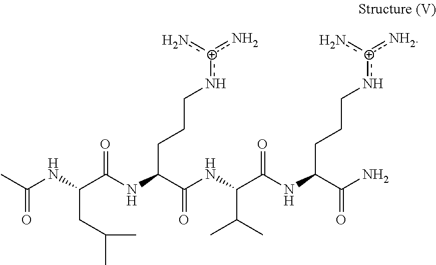

For the peptide compound that begins with acetyl-LRK, i.e., SEQ ID NO: 17, the non-acctylated version is shown below in structure (VI):

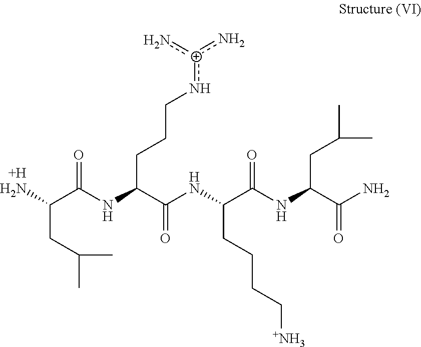

The acetyl-LRK structure of SEQ ID NO: 17 is shown below in structure (VII):

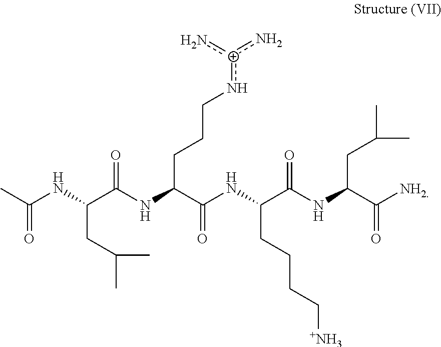

In peptide compounds SEQ ID NOs: 2-3 and 19, the amino terminal amino acid is bonded to picolinyl (i.e., picolinyl-CONH—). The amino terminus of SEQ ID NOs: 2-3 begins with alanine-serine and the amino terminus of SEQ ID NO: 19 begins with leucine-leucine. The schematic structures shown below are for alanine-serine, but the same type of bond to the picolinyl group is present in SEQ ID NO: 19. The free amino group (i.e., no picolinyl group) of SEQ ID NOs: 2-3 is shown below in structure (VIII):

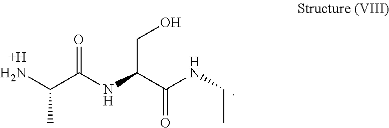

The picolinyl modified amino terminus, (i.e., picolinyl-AS of SEQ ID NOs: 2-3) is shown below in structure (IX):

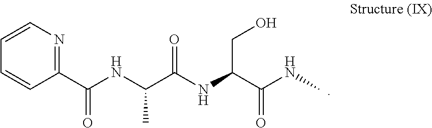

In peptide compound SEQ ID NO: 13, the amino terminal amino acid is bonded to “C8” which is to be understood as meaning an eight straight-chain carbon atom octanyl group. The free amino terminus of SEQ ID NO: 13 is shown below in structure (X):

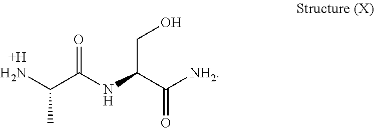

The addition of the C8 group on the amino terminus of SEQ ID NO: 13 is shown below in structure (XI):

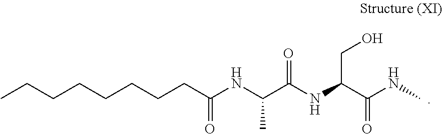

In peptide compound SEQ ID NO: 14, the epsilon amino group of the amino terminal amino acid (lysine) is bonded to an eight straight-chain carbon atom group (i.e., C8 group) and the alpha amino group of the amino terminal amino acid (lysine) is bonded to an acetyl group, which is shown below in structure (XII):

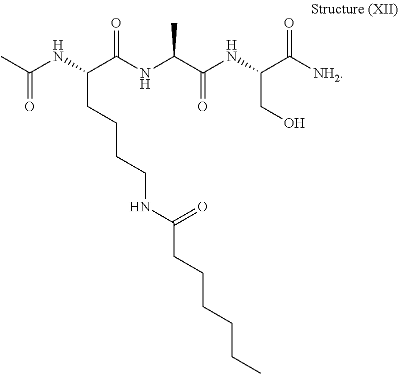 [SMILES: CCCCCCC(=O)NCCCC[C@H](NC(C)=O)C(=O)N[C@@H](C)C(=O)N[C@@H](CO)C(N)=O]

In peptide compound SEQ ID NO: 15, the epsilon amino group of the amino terminal amino acid (lysine) is bonded to a picolinyl group and the alpha amino group of the amino terminal amino acid (lysine) is bonded to an acetyl group, which is shown below in structure (XIII):

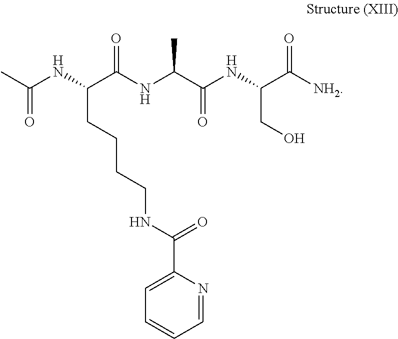 [SMILES: CC(=O)N[C@@H](CCCCNC(=O)c1ccccn1)C(=O)N[C@@H](C)C(=O)N[C@@H](CO)C(N)=O]

### Method of Preparing the Peptide Compounds of the Present Invention

The following discussion provides principles for obtaining the peptide compounds of the present invention and gives details of some methods available for preparing the peptide compounds of the present invention. However, the discussion is not intended to define or limit the scope of reactions or reaction sequences that can be used in the preparation of peptide compounds of the invention. The peptide compounds of the present invention can be prepared by the steps and techniques disclosed in the Examples section herein below and known organic synthesis techniques. The peptide compounds of the present invention can be synthesized with conventional solid phase or liquid phase peptide synthesis and qualified by HPLC and Mass Spectrometry as known to those skilled in the art.

Methods known to those of ordinary skill in the art can be confirmed through various reference books and published data. Detailed description of the available reactants in the synthesis of the compounds of the present invention are available, or provided in references described in the literature and appropriate reference books including, for example, “Practical Synthetic Organic Chemistry”, John Wiley & Sons, Inc., New York 2011; and the SR Sandler et al., “Organic Functional Group Preparations”, 2nd Ed, Academic Press, New York, 1983; the H. O. House, “Modern Synthetic Reactions”, 2nd Ed., WA Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structures”, 4th Ed, Wiley.—Interscience, New York, 1992. A detailed description of the synthesis of reactants that can be used in the preparation of the compounds of the invention or other suitable reference books and monographs that provide references to the literature describing the preparation methods include, for example, Fuhrhop, J. and Penzlin G. “Organic Synthesis: Concepts, Methods, Starting Materials”; Second, Revised and Enlarged Edition (1994) John Wiley & Sons ISBN: 3-527-29074-5; Hoffman, RV “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; Otera, J. (editor) “Modern Carbonyl Chemistry” (2000) Wiley-VCH, ISBN: 3-527-29871-1; Patai, S. “Guide to The Chemistry of Functional Groups” (1992) Interscience ISBN: 0-471-93022-9; Quin, L. D. et al., “A Guide to Organophosphorus Chemistry” (2000) Wiley-Interscience, ISBN: 0-471-31824-8; Solomons, T. W. G. “Organic Chemistry” 7th Edition (2000) John Wiley & Sons, ISBN: 0-471-19095-0; Stowell, J. C. “Intermediate Organic Chemistry” 2nd Edition (1993) Wiley-Interscience, ISBN: 0-471-57456-2; “Ullmann's Encyclopedia: Industrial Organic Chemicals: Starting Materials and Intermediates” (1999) John Wiley & Sons, ISBN: 3-527-29645-X-, in Volume. 8 “Organic Reaction” (1942-2000) John Wiley & Sons, in over 55 Volumes; and “Chemistry of Functional Groups” John Wiley & Sons, in 73 Volumes.

In summary, the compounds used in the reactions described herein can be prepared from commercially available chemical reagents and/or compounds described in the chemical literature according to organic synthesis techniques known to those skilled in the art. “Commercially available chemical reagents” can be obtained from standard commercial sources including Acros Organics (Pittsburgh PA), Aldrich Chemical (Milwaukee WI, including Sigma Chemical and Fluka), Apin Chemicals Ltd. (Milton Park UK), Avocado Research (Lancashire UK), BDH Inc. (Toronto, Canada), Bionet (Cornwall, UK), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall UK), Lancaster Synthesis (Windham NH), Maybridge Chemical Co. Ltd. (Cornwall UK), Parish Chemical Co. (Orem UT), Pfaltz & Bauer, Inc. (Waterbury CN), Polyorganix (Houston TX), Pierce Chemical Co. (Rockford IL), Riedel de Haen AG (Hanover, Germany), Spectrum Quality Product, Inc. (New Brunswick, NJ), TCI America (Portland OR), Trans World Chemicals, Inc. (Rockville MD) and Wako Chemicals USA, Inc. (Richmond VA).

Specific reactants and similar reactants can be confirmed by an index of known chemical reagents (obtained from most public or university libraries) prepared by the Chemical Abstracts Service of the American Chemical Society or through online databases. Chemical reagents that are known but not commercially available in the catalog can be prepared by custom chemical reagent synthesizers, many of which are standard chemical reagent providers (such as those above) that provide custom synthesis services.

For example, the way of forming —SS— between amino acids within the cyclic peptide compound SEQ ID NO: 3 of the present invention is known to those skilled in the art, and can be found in, for example, Pohl, M., et al, J. Peptide Protein Res. 41 (4): 362-375. (1993); Tam, J P, et al, J ACS. 113 (17): 6657-6662. (1991); and in Example 1 of the present disclosure, which describes a method of preparation of the peptide compounds.

In some aspects of the invention, an ApoE-based synthetic peptide Acetyl-AS-aib-LRKL-aib-KRLL-amide (SEQ ID NO: 1) designated “COG1410” is provided for use as an antimicrobial. In one embodiment, the peptide COG1410 is provided for use in a method for inhibiting gram-negative bacteria including Enterobacter cloacae, Escherichia coli, Citrobacter freundii and even anaerobe, Porphyromonas gingivalis with MICs ranging from 16 to 64 μg/ml (see Examples 1 & 2 herein and Table 1 below). In another embodiment, the peptide compound COG1410 is provided for use in a method for inhibiting the pan-drug resistant bacteria Acinetobacter baumannii YQ4. Remarkably and unexpectedly, the experiments described in Examples 1-3 show that COG1410 can kill Acinetobacter baumannii YQ4, with minimal inhibitory concentration (MIC) and minimal bactericidal concentration (MBC) 16 μg/ml (11.3 μM). In addition, COG1410 was found to inhibit 107 other clinically collected A. baumannii strains with MICs ranging from 16 to 32 μg/ml.

In other aspects of the invention, the synthetic peptide compounds of the invention are provided for use as antimicrobials. The MICs for the peptide compounds are shown in Table 1 against E. coli, L. salivarius, and P. gingivalis. Determination of the MIC values in Table 1 is described in Example 3.

TABLE 1

| | | | MIC | MIC |
|---|---|---|---|---|
| SEQ | | MIC | L. | P. |
| ID | | E. coli | salivarius | gingivalis |
| NO: | Peptide compound | (ug/ml) | (ug/ml) | (ug/ml) |
| 1 | Acetyl-AS-aib-LRKL-aib-KRLL-amide | | | 16-64 |
| 2 | Picoliny1-AS-aib-LRKL-aib-KRLL-amide | 32 | 8 | 2-32 |
| 3 | Picolinyl-AS-C-LRKL-aib-KRLL-C-amide | | | 2 |
| | having a disulfide link between | | | |
| | the two cysteines | | | |
| 4 | Acetyl-LLRK(Aib)LKRL(Aib)SA-CONH2 | 8 | 8 | 32 |
| 5 | Acetyl-llrk(Aib)Ikkl(Aib)sa-amide | 16 | | 16 |
| | (small letters are D amino acids) | | | |
| 6 | Acetyl-as(Aib)lrkl(Aib)krll-amide | 8 | 8 | 8 |
| | (small letters are D amino acids) | | | |
| 7 | Acetyl-LLRK-Aib-LRKL-Aib-SAS-Aib-LRKL- | 8 | 8 | 16 |
| | Aib-KRLL-CONH2 | | | |
| 8 | Acetyl-LRVRCAS(Aib)LRKL(Aib)KRLL- | 8 | 16 | 64 |
| | CONH2 | | | |
| 9 | Acetyl-LRVRLAS(Aib)LKKL(Aib)KRLL-Amide | 4 | 4 | 8 |
| 10 | Acetyl -LRVRLAS(Aib)LRKL(Aib)KRLL-Amide | | | |
| 11 | Acetyl -llrk(aib)lkrl(aib)salrvrl-amide | 4 | 16 | 8 |
| | (small letters are D amino acids) | | | |
| 12 | Acetyl-LR VRLASHLRKLRKRLLAS-aib-LRKL- | 4 | 64 | 16 |
| | aib-KRLL-CONH2 | | | |
| 13 | C8-AS-aib-LRKL-aib-KRLL-amide | | | |
| 14 | Acetyl-K(C8)-AS-aib-LRKL-aib-KRLL-amide | | | |
| 15 | Acetyl-K(Picoliny1)-AS-aib-LRKL-aib- | | | |
| | KRLL-amide | | | |
| 16 | Acetyl-LRVRLASHLRKLRKRLLR-amide | | | |
| 17 | Acetyl-LRKLRKRLLLRKLRKRLL-amide | | | |
| 18 | Acetyl- | | | |
| | LRVRLASHLRKLRKRLLRDADDLQKRLAVY- | | | |
| | amide | | | |
| 19 | Picolinyl-llrk-aib-lkrl-aib-salrvrl- | | | |
| | amine (small letters are D-amino acids) | | | |

“Aib” in Table 1 is amino isobutyric acid

COG1410 contains amino acid residues located between residues 138-149 of the ApoE N-terminal domain with amino isobutyric acid (Aib) substitutions at positions 140 and 145. COG1410 is a modification of a peptide based on residues 133-149 of ApoE to extend the therapeutic window of post-TBI treatments, and has demonstrated neuroprotective activity in several models of brain injuries, including intracerebral hemorrhage and focal brain ischemia. Through reducing inflammation and apoptosis, COG1410 enhances retinal ganglion cell survival and alleviates early brain injury. Furthermore, COG1410 has been shown to possess the ability to target the blood brain barrier (BBB). COG1410 has been fused with Aβ binding region to form a multi-strategy peptide, which enhanced the BBB targeting efficiency and ameliorated neurologic damage in the mice Alzheimer's disease model. Therefore, COG1410 has been considered as a promising therapeutic agent for diseases related to neural injury.

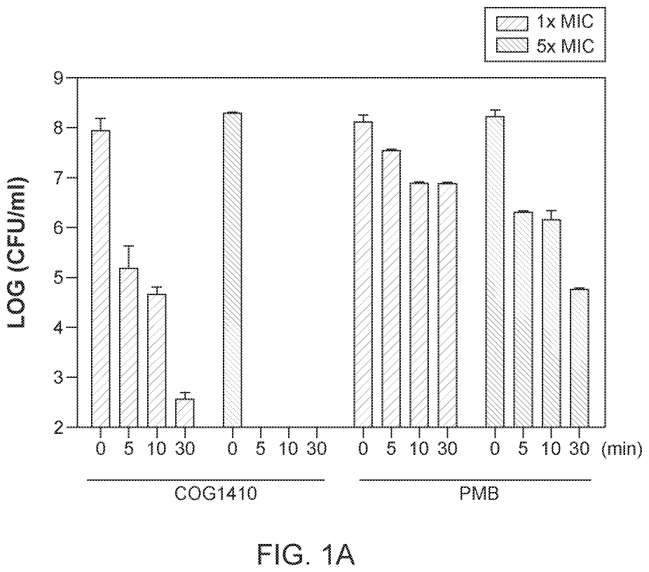

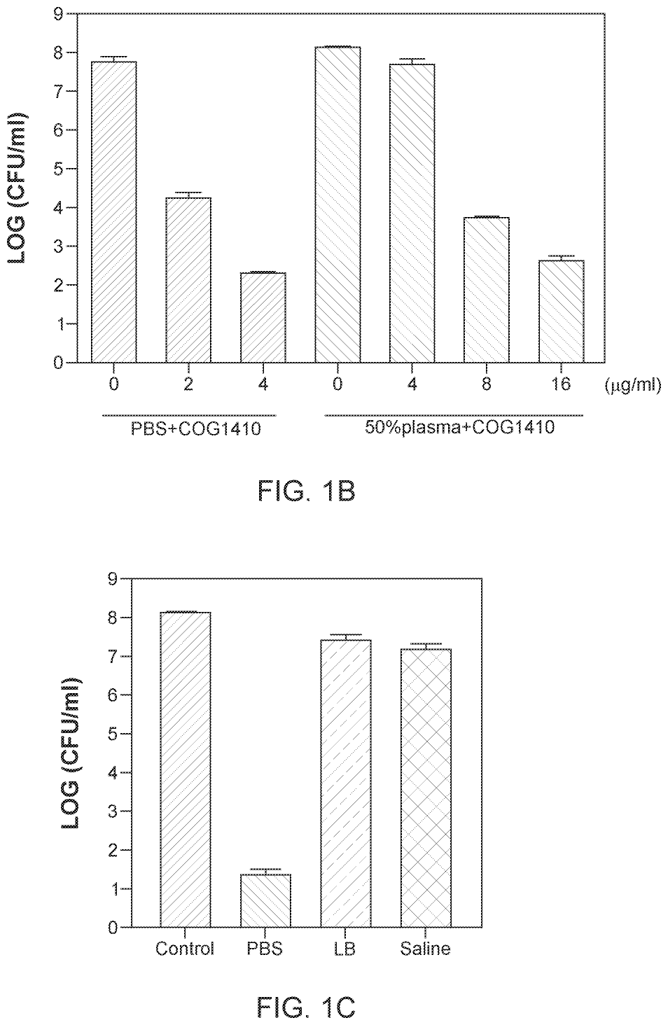

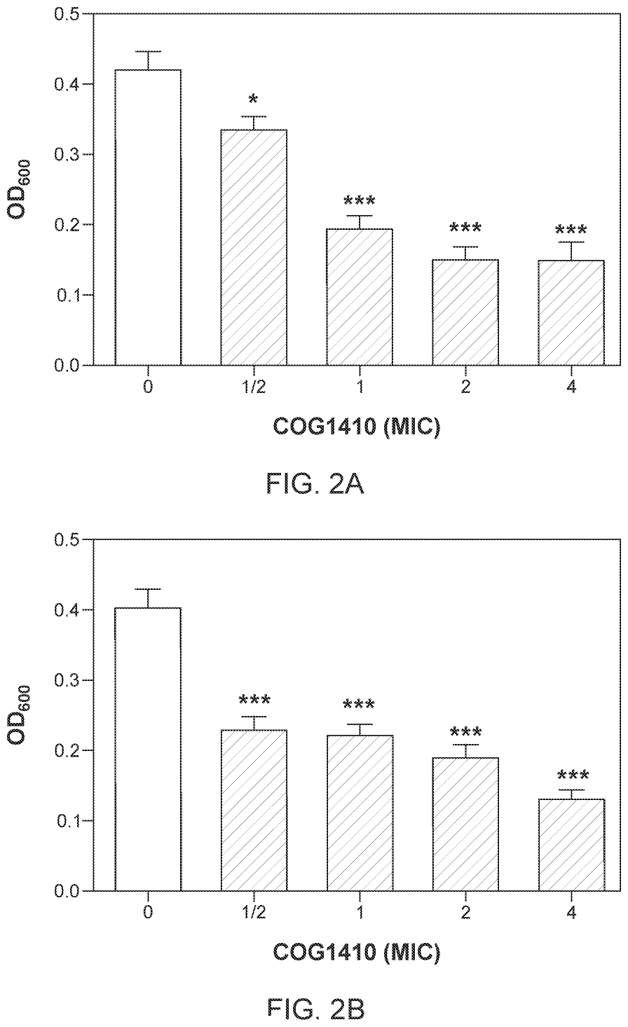

In some embodiments of the invention, COG1410 is provided for use as an antimicrobial agent. Experimental data provided herein in Examples 1-3 and FIGS. 1-8 illustrate the antibacterial efficacy of COG1410. Specifically, COG1410 exhibited broad spectrum antibacterial and potent bactericidal activity, especially against strains of the pandrug-resistant bacterium, Acinetobacter baumannii. COG1410 took effect very rapidly in vitro. For example, 1×MIC COG1410 reduced CFU of A. baumanii by 3 logs (1000 fold) within 5 min, which was much faster than polymyxin B (PMB) (see FIG. 1A), and equivalent to that of the promising anti-A. baumannii antimicrobial peptide, ZY4, which is a cathelicidin-derived peptide that kills A. baumannii within 30 min at 1×MIC [24]. FIGS. 2A & 2B show that COG1410 can inhibit biofilm formation and eradicate mature biofilm in A. baumannii. A. baumannii is one of the major biofilm-producing bacteria and due to its biofilm formation easily survives and spreads in the hospital environment. Therefore, in one embodiment, COG1410 is provided for use as an antimicrobial against A. baumannii.

One major challenge for therapeutic application of antimicrobial peptides is degradation or inactivation in plasma. The bactericidal efficacy of COG1410 was assessed in plasma. The LC_{99.9} of COG1410 in PBS and 50% pooled plasma were 1.4 μM and 5.6 μM, respectively (see FIG. 1B). This was better than LL-37-derived AMP, SAAP-148, of which the corresponding LC_{99.9} were 1.6 μM and 12.8 UM against A. baumannii. In addition, stability studies show that COG1410 can be very stable in plasma. Specifically, COG1410 was not significantly degraded in 100% human plasma within 2 h. Even after 10 h, more than 80% of activity was retained. Consistent with these results, COG1410 has been administered via intravenous injection in a murine model of traumatic brain injury (TBI). In that study, a single intravenous injection of COG1410 significantly improved vestibulomotor function and spatial learning and memory. Similar effects were observed in a rat model of focal brain ischemia and mouse model of Traumatic optic nerve injury. Therefore, in one embodiment, COG1410 is provided for administration to a subject as an antimicrobial for systemic infection.

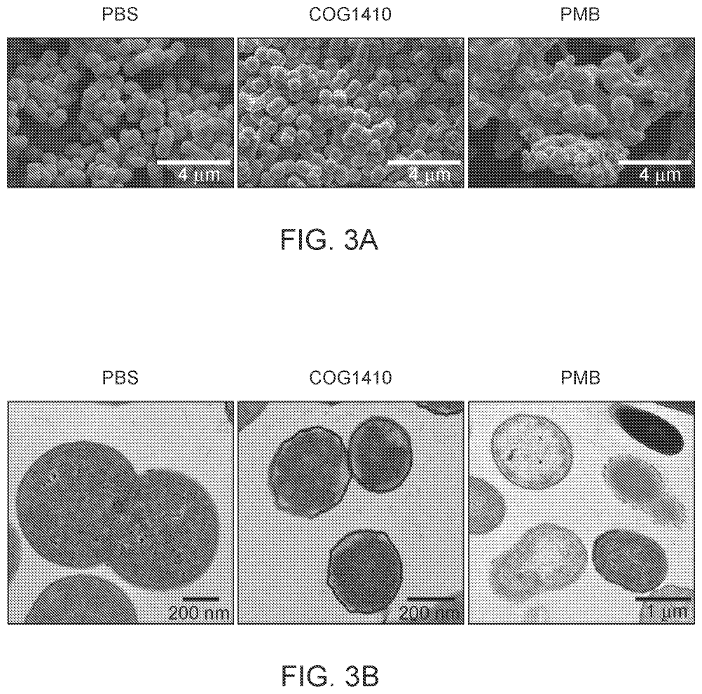

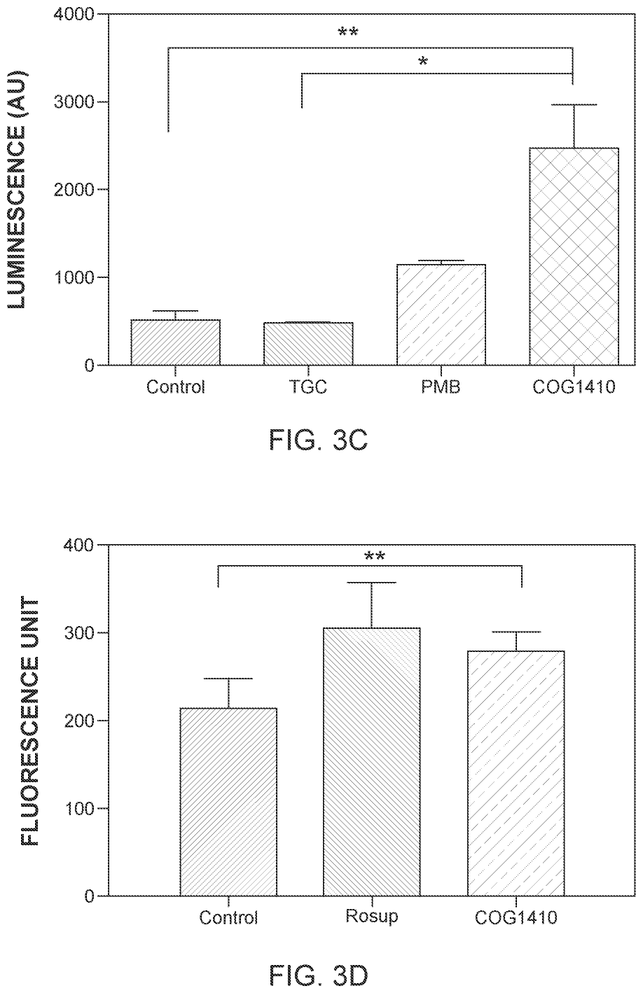

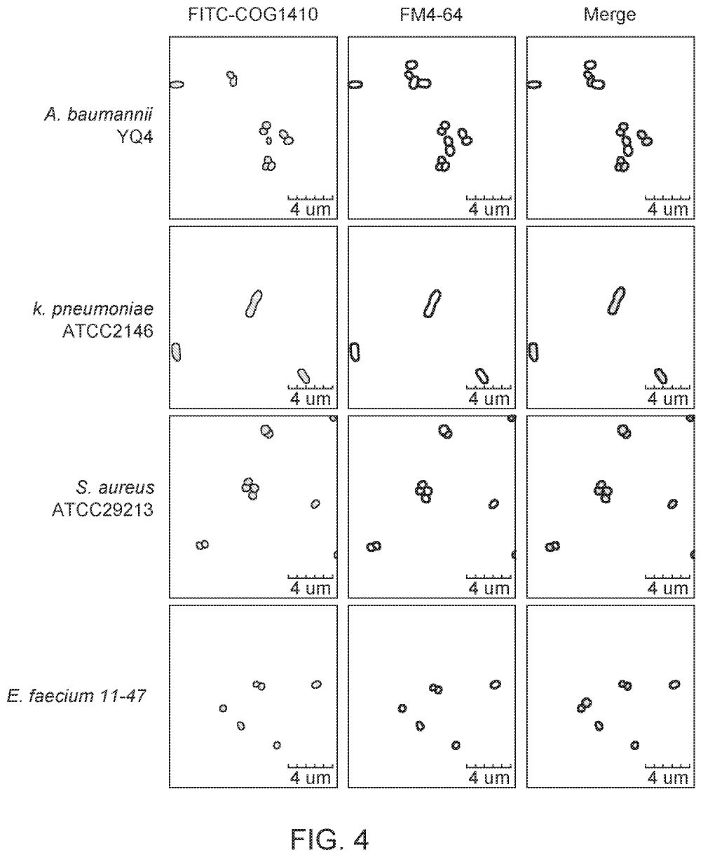

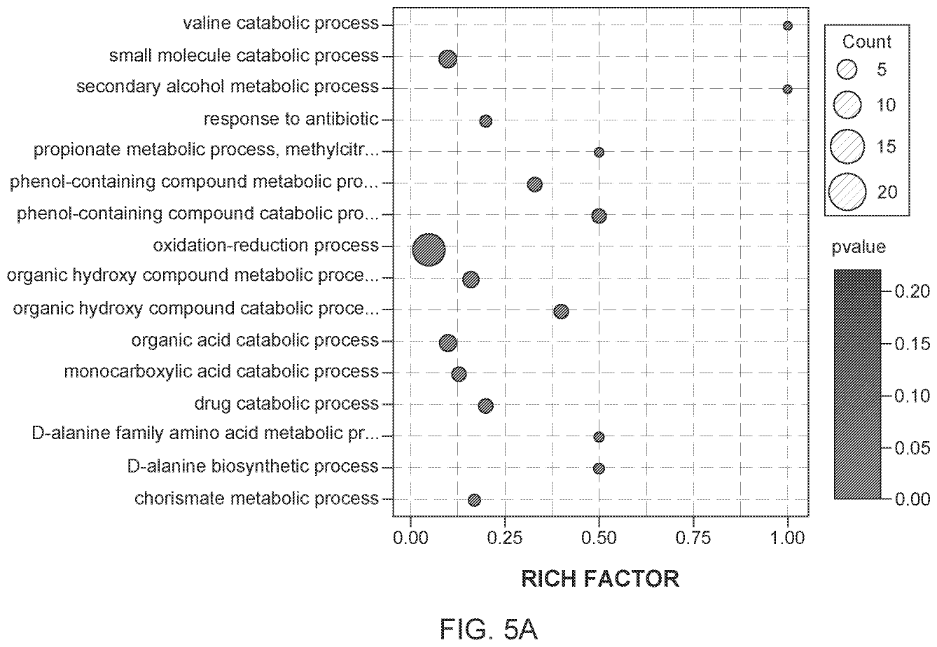

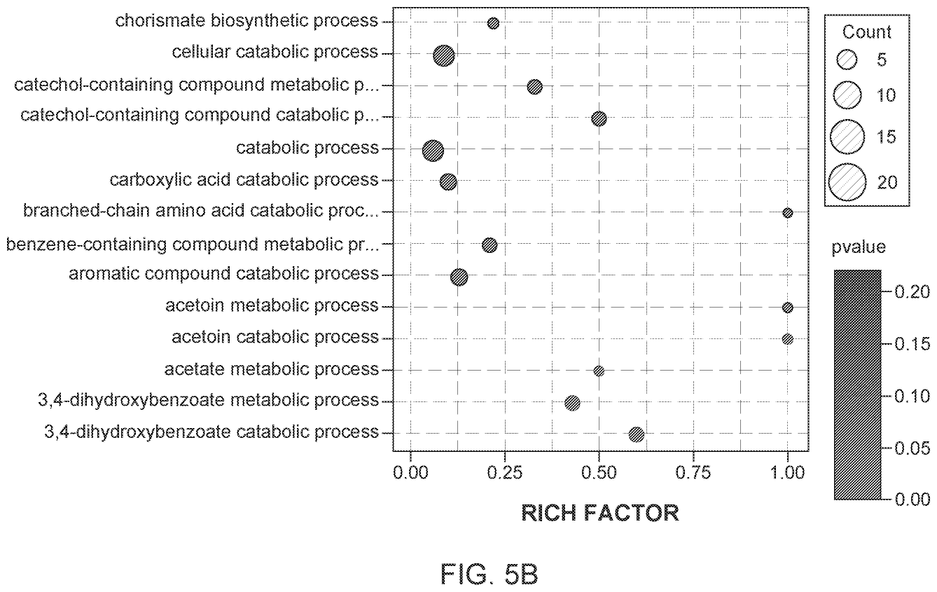

Most cationic AMPs like COG1410 directly target the cell membrane, causing pore formation and final cell lysis. As expected, the experiments described in Example 2 show that treatment of polymyxin B leads to collapse of the cell wall and release of cytoplasmic content (see e.g., FIG. 3A-3D and FIG. 4). However, unexpectedly, cells treated with COG1410 remained intact without pore formation. Cell death may have been due to the separation between inner membrane and cell wall. Significant ATP leakage by cells exposed to COG1410 was observed in the experiments described in Example 2, which indicates that the cell membrane was disrupted or permeabilized. Consistently, fluorescent dye staining assays also confirmed this conclusion. On the other hand, it was observed that COG1410 could cross the cell membrane and enter the cytoplasm of A. baumannii. RNA-seq analysis revealed that sub-MIC levels of COG1410 significantly affected genes of A. baumannii in minimal medium, in which genes in the oxidative and reductant biological process were remarkably enriched (see FIG. 5). DCFH-DA probe also detected 30% more ROS after COG1410 treatment for 20 min. These data suggest that COG1410 can induce oxidative stress in A. baumannii. Experiments described in Example 2 suggest that COG1410 can bind DNA nonspecifically. One theory previously set forth in the literature is that membrane disruption and DNA binding are two hits against bacteria by the AMP, NK18. However, COG1410 was able to enter the cytoplasm of P. aeruginosa, S. aureus and E. faecium, but these bacteria were not sensitive to the antimicrobial activity. Therefore, and without wishing to be bound to any particular mechanism of action, DNA binding might not play a key role in the mechanism of COG1410's antimicrobial activity. Again, not being limited to any one mechanism of action, the data taken together show that COG1410 may inhibit bacterial growth by a mechanism that includes one or both of disruption of the integrity of cell membrane and induction of oxidative stress.

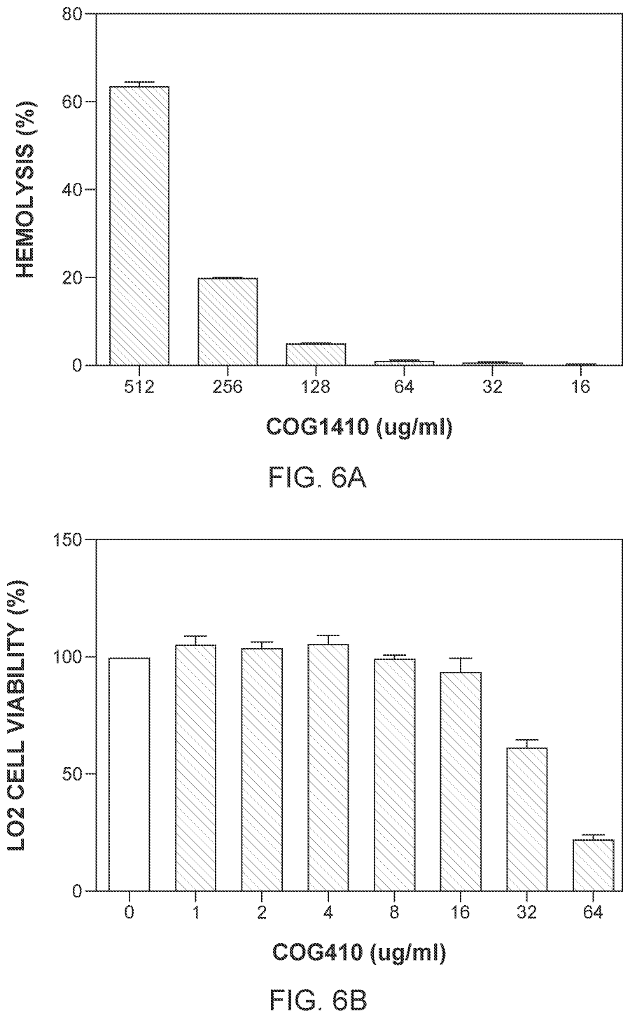

As a class of drugs, cationic AMPs may be particularly troublesome with regard to cytotoxicity. It was observed in the experiments described in Example 2 that 128 μg/ml (8×MIC in LB) COG1410 results in less than 5% hemolysis (FIG. 6A). The cytotoxic effect of COG1410 on normal human hepatic L02 cells was evaluated by CCK8 assay as described in Example 2. The EC₅₀ of COG1410 was 58.9 μg/ml for the L02 cell (FIG. 6B). In addition, the LC_{99.9} was 2 μg/ml and 8 μg/ml in PBS and 50% plasma, respectively. These results indicate an improved cytotoxicity profile for COG1410 relative to many existing antimicrobial peptides.

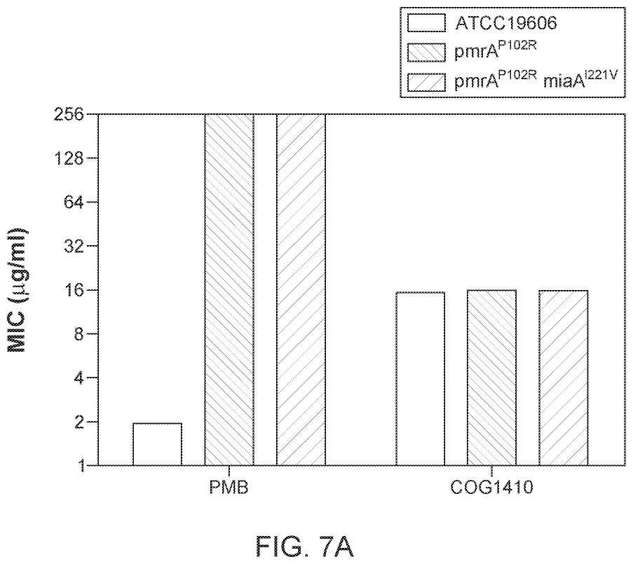

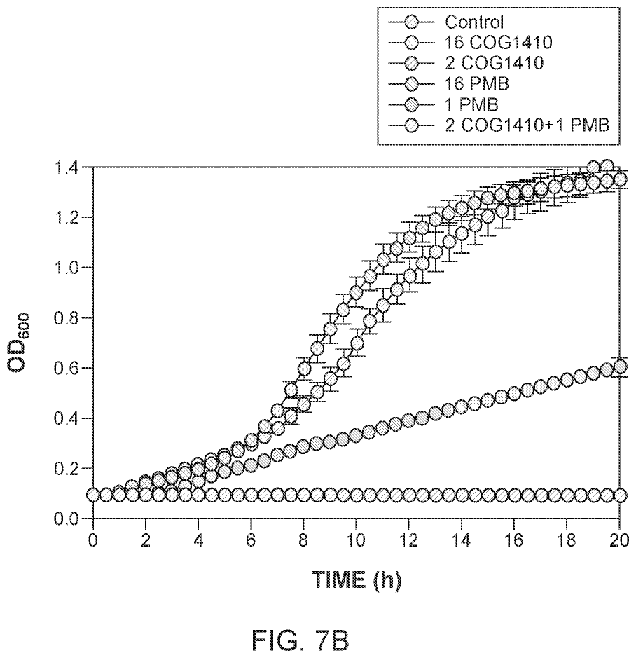

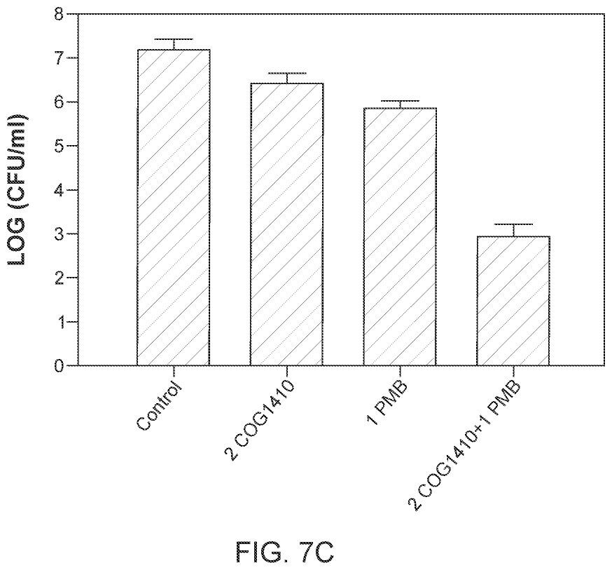

Another advantage of the use of COG1410 as an antimicrobial is that a significant synergistic interaction was observed for the combination of COG1410 and polymyxin B. Specifically, the working concentration was reduced to 2 μg/ml for COG1410 and 1 μg/ml for polymyxin B (FIG. 7A-7C). In addition, it was noticed that the bactericidal effect of COG1410 did not depend on the electrostatic interaction with LPS, since modification of LPS did not inhibit the activity of COG1410. This feature contrasts with what has been previously observed for polymyxin B and LL-37, where the initial step in attacking the bacterial pathogen is binding with LPS. It is generally recognized that polymyxin B binds with the lipid A portion of the LPS in Gram-negative bacteria, replacing cationic ions such Ca²⁺ and Mg²⁺, which destabilizes the LPS layer and the membrane.

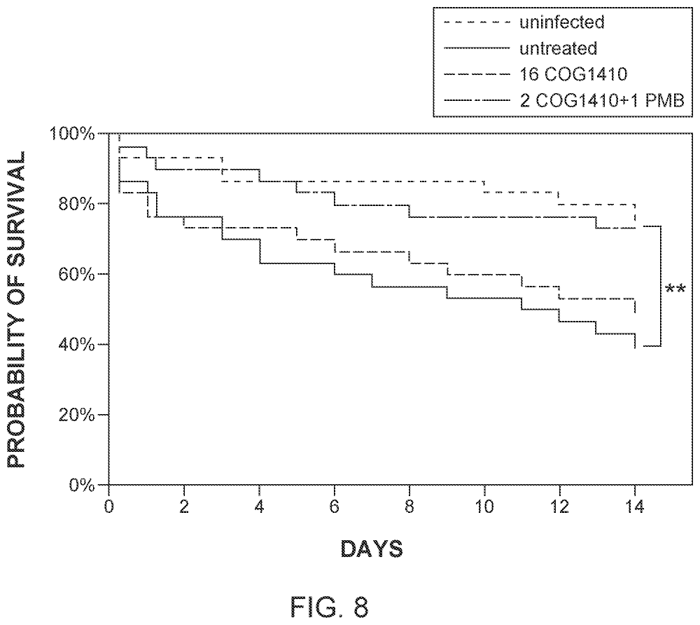

In another experiment, the graph in FIG. 8 illustrates that the combined therapy of COG1410 and polymyxin B rescued infected nematodes. C. elegans were pre-infected by A. baumannii YQ4 and transferred to a NGM plate supplemented with 16 μg/ml COG1410 or 2 μg/ml COG1410 and 1 μg/ml polymyxin B. The dead nematodes were counted every day for 2 weeks. The data in FIG. 8 show the unexpected result that the combination therapy of COG1410 (2 μg/ml) plus polymyxin B (1 μg/ml) significantly rescued the pre-infected nematodes and even elongated their lives.

Combination drug therapy is used frequently in medical practice to treat microbial infections, especially in the intensive care unit. Therefore, the synergistic activity between polymyxin B and COG1410 is another advantage provided for using COG1410 in combination with polymyxin B to treat microbial infections.

The experimental results provided in Example 2 show the potent antibacterial capability of the ApoE mimetic peptide, COG1410. The major bactericidal mechanism of COG1410 was to disrupt cell membrane integrity and induce oxidative stress. COG1410 displayed strong bacterial killing, high stability in human plasma and low propensity for resistance development. The synergistic interaction between COG1410 and polymyxin B reduces the working concentration of COG1410 and avoids its risk of eukaryotic cell toxicity. Considering that it simultaneously has neuroprotective, anti-inflammation and antibacterial effects, COG1410 is provided, in one aspect of the invention, in a method for treating a subject having a microbial infection. The method includes administering to the subject a therapeutically effective amount of a composition comprising the peptide compound COG1410, or a pharmaceutically acceptable salt or solvate thereof, to inhibit microbial growth in the subject. The microbial growth can be growth of pandrug-resistant Acinetobacter baumannii, which infection has been considered a health-care crisis.

In one aspect of the invention, a method is provided for inhibiting microbial growth, comprising: contacting a microbe with an effective amount of a peptide compound of the invention to inhibit the microbial growth, wherein the peptide compound of the invention is selected from SEQ ID NOs: 1-19. In some embodiments, the peptide compound comprises SEQ ID NO: 1, SEQ ID NO: 2, SEQ ID NO: 3, SEQ ID NO: 13, SEQ ID NO: 14, or SEQ ID NO: 15. In other embodiments, the peptide compound comprises SEQ ID NO: 1, SEQ ID NO: 2, or SEQ ID NO: 3. In some cases, the peptide compound comprises SEQ ID NO: 1.

In the method, the microbe can include a bacterium that is one or more of multidrug-resistant (MDR), extensively drug-resistant (XDR), or pandrug-resistant (PDR). In some embodiments, the microbe includes Porphoryomas gingivalis. The microbes can include a Gram-negative bacterial pathogen or a Gram-positive pathogen. The Gram-negative bacterial pathogens can include one or a combination of Acinetobacter baumannii, P. gingivalis, or E. coli and the Gram-positive pathogens can include one or a combination of S. aureus or L. salivarius.

In the method, the peptide compound can be contacted in combination with one or more other antimicrobial compounds. In one example, the one or more other antimicrobial compounds can include polymyxin B.

In some embodiments, the peptide compound can be contacted in combination with polymyxin B to inhibit a microbe comprising pandrug-resistant Acinetobacter baumannii or multidrug-resistant S. aureus.

In some embodiments, the peptide compound can be contacted in combination with polymyxin B to inhibit a microbe comprising Acinetobacter baumannii, wherein the peptide compound comprises: SEQ ID NO: 1, SEQ ID NO: 2, SEQ ID NO: 3, SEQ ID NO: 13, SEQ ID NO: 14, or SEQ ID NO: 15. In other instances, the peptide compound can be contacted in combination with polymyxin B to inhibit a microbe comprising Acinetobacter baumannii, wherein the peptide compound comprises: SEQ ID NO: 1, SEQ ID NO: 2, or SEQ ID NO: 3. In one instance, the peptide compound can be contacted in combination with polymyxin B to inhibit a microbe comprising Acinetobacter baumannii, wherein the peptide compound comprises SEQ ID NO: 1.

The peptide compounds of the present invention, SEQ ID NOs: 1-19, share a similarity to the receptor binding region of the holo-ApoE protein. Therefore, all the peptide compounds of the present invention can be expected to exhibit similar antimicrobial activity.

In addition, peptide compounds SEQ ID NOs: 1 and 2 share a similar structure and only differ at the amino terminus. Specifically, SEQ ID NO: 1 has an acetyl group at the amino terminus and SEQ ID NO: 2 has a picolinyl group at the amino terminus. As described above and shown in Table 1, SEQ ID NO: 1 has excellent antimicrobial activity against Acinetobacter baumannii and is also effective against P. gingivalis. SEQ ID NO: 2 is shown to be even more effective against P. gingivalis than SEQ ID NO: 1 and to also have antimicrobial activity against E. coli and L. salivarius (see Table 1). Therefore, the antimicrobial activity of SEQ ID NO: 2 against pathogens such as Acinetobacter baumannii can be expected to be similar to that of SEQ ID NO: 1. SEQ ID NO: 3 has the same structure as SEQ ID NO: 2 with the exception that the Aib groups are replaced by cysteines which form a disulfide bond. Like SEQ ID NO: 2, SEQ ID NO: 3 displays high antimicrobial activity against P. gingivalis (see Table 1). Therefore, the antimicrobial activity of SEQ ID NO: 3 against pathogens such as Acinetobacter baumannii can also be expected to be similar to that of SEQ ID NO: 1.

Peptide compounds SEQ ID NOs: 13, 14 and 15 have a similar structure as SEQ ID NOS: 1 and 2 and only differ at the amino terminus. Specifically, SEQ ID NO: 13 has a C8 group at the amino terminus, SEQ ID NO: 14 has an acetyl-K (C8) group at the amino terminus, and SEQ ID NO: 15 has an acetyl-K (Picolinyl) group at the amino terminus. For the reasons described above, SEQ ID NOs: 13, 14 and 15 can be expected to have similar antimicrobial activities as SEQ ID NOs: 1-3.

The invention includes a method for treating a subject having a microbial infection, the method including administering to the subject a therapeutically effective amount of a pharmaceutical composition comprising a peptide compound of the present disclosure, or a pharmaceutically acceptable salt or solvate thereof, to inhibit microbial growth in the subject. The subject can be a mammal, a primate, or a human.

In various embodiments, the microbial growth can be caused by one or more bacteria that can be multidrug-resistant (MDR), extensively drug-resistant (XDR), or pandrug-resistant (PDR).

In other embodiments, the microbial growth can be caused by an oral infection with a bacterium that includes Porphoryomas gingivalis.

The administering of the pharmaceutical composition comprising the peptide compound to the subject can include a single administration or multiple administrations of the pharmaceutical composition. The pharmaceutical composition comprising the peptide compound can be administered topically, enterally, systemically, or parenterally. In some instances, the pharmaceutical composition comprising the peptide compound can be administered in a formulation of a mouth wash or a toothpaste. In some cases when the microbial growth is caused by an oral infection with a bacterium that includes Porphoryomas gingivalis, the pharmaceutical composition comprising the peptide compound can be administered in a formulation of a mouth wash or a toothpaste.

In some embodiments of the uses or methods described herein, the dose of the peptide compound of the present invention generally depends on a variety of factors, including the severity of the individual or microbial infection being treated, the rate of administration, and the judgment of the prescribing physician. In general, the effective daily dose per kg body weight can range from about 0.01 to about 1.0 mg, for example, about 0.01 to about 1.0, 0.01 to about 0.1, 0.01 to about 0.09, 0.01 to about 0.08, 0.01 to about 0.07, 0.01 to about 0.06, 0.01 to about 0.05, or 0.01 to about 0.04 mg/kg/day. In one embodiment, the dose is 0.051 mg/kg/day on the first day followed by 0.017 mg/kg/day on subsequent days. The precise dose may be varied on each day of dosing to achieve the desired therapeutic efficacy.

In the method of treating, the bacterium being inhibited can be a gram-negative or a gram-positive pathogen. In the case of gram-negative bacteria, the bacterium can include, but is not limited to, one or a combination of Acinetobacter baumannii, P. gingivalis, and E. coli. In the case of gram-positive bacteria, the bacterium can include, but is not limited to, one or a combination of S. aureus and L. salivarius.

In other aspects of the invention, the pharmaceutical composition comprising the peptide compound of the present disclosure is administered in combination with one or more other antimicrobial compounds. For example, the one or more antimicrobial compounds can include polymyxin B.

In one instance, the pharmaceutical composition comprising the peptide compound (COG1410) acetyl-AS-aib-LRKL-aib-KRLL-amide (SEQ ID NO:1) is administered in combination with polymyxin B to synergistically inhibit the pandrug-resistant bacterium Acinetobacter baumannii.

In other instances, the pharmaceutical composition comprises the peptide compound SEQ ID NO: 1, SEQ ID NO:2, or SEQ ID NO:3, and the pharmaceutical composition is administered in combination with polymyxin B to inhibit Acinetobacter baumannii.

In another instance, the pharmaceutical composition comprising the peptide compound SEQ ID NO: 1, SEQ ID NO: 2, SEQ ID NO: 3, SEQ ID NO: 13, SEQ ID NO: 14, or SEQ ID NO: 15 is administered in combination with polymyxin B to inhibit Acinetobacter baumannii.

In some embodiments, the pharmaceutical composition comprising the peptide compound is administered in combination with one or more antimicrobial compounds to inhibit Acinetobacter baumannii or multidrug-resistant S. aureus. The pharmaceutical compositions of the invention include an antimicrobial composition including: (i) a therapeutically effective amount of a peptide compound comprising SEQ ID NO: 1, SEQ ID NO: 2, SEQ ID NO: 3, SEQ ID NO: 13, SEQ ID NO: 14, or SEQ ID NO: 15; and (ii) a therapeutically effective amount of polymyxin B. In some embodiments, the peptide compound in the antimicrobial composition comprises SEQ ID NO: 1, SEQ ID NO: 2, or SEQ ID NO: 3. In other instances, the peptide compound in the antimicrobial composition comprises SEQ ID NO: 1.

In one embodiment, the antimicrobial composition including a peptide compound of the invention is provided for use in a method of inhibiting microbial growth in a subject. In another embodiment, a pharmaceutical composition including the antimicrobial composition is provided for use in a method of inhibiting microbial growth in a subject.

The peptide compounds of the present disclosure include pharmaceutically acceptable salts or solvates thereof, for use in the method of inhibiting microbial growth in a subject.

Pharmaceutical compositions are provided comprising a peptide compound of the present disclosure, or a pharmaceutically acceptable salt or solvate of a peptide compound of the present disclosure.

In one embodiment, the pharmaceutical composition includes a peptide compound: SEQ ID NO: 1, SEQ ID NO: 2, SEQ ID NO: 3, SEQ ID NO: 13, SEQ ID NO: 14, or SEQ ID NO: 15. In other embodiments, the pharmaceutical composition includes a peptide compound: SEQ ID NO: 1, SEQ ID NO: 2, or SEQ ID NO: 3. In one instance, the peptide compound included in the pharmaceutical composition comprises SEQ ID NO: 1.

The pharmaceutical compositions of the present disclosure comprising the peptide compound can include one or more other antimicrobial compounds. For example, the antimicrobial compound can comprise polymixin B.

In one embodiment, the pharmaceutical compositions of the present disclosure are provided for use in a method of inhibiting microbial growth in a subject.

Administering the pharmaceutical compositions of the present invention to a subject can include a single administration or multiple administrations of the pharmaceutical composition. The pharmaceutical composition comprising the peptide compound can be administered topically, enterally, systemically, or parenterally. In some instances, the pharmaceutical composition can be administered in a formulation of a mouth wash or a toothpaste. In some cases when the microbial growth is caused by an oral infection with a bacterium that includes Porphoryomas gingivalis, the pharmaceutical composition comprising the peptide compound can be administered in a formulation of a mouth wash or a toothpaste.

The pharmaceutical composition may include one or more pharmaceutically acceptable excipients. The one or more excipients may include, but is not limited to, disintegrants, diluents, binders, solvents, co-solvents, lubricants, pH adjusting agents, buffering agents, preservatives, dispersing agents, suspending agents, ointment bases, emulsifiers, emollients, penetration agents, surfactants, propellants, flavoring agents, sweetening agents, or drug release modifiers. The pharmaceutical composition may include a solvent such as one or both of physiological saline and glucose solution.

The pharmaceutical compositions of the invention may be formulated as a freeze-dried powder or as a liquid suitable for administration by injection, to cause slow release of the peptide compound, or as a spray. In other embodiments, the pharmaceutical compositions of the invention may be formulated as a mouth wash or as a toothpaste.

The present invention will be described in more detail below through examples, but these examples are not intended to limit the present disclosure.

### EXAMPLES

### Example 1. Peptide Synthesis, Purification and Identification

COG1410 was synthesized with conventional solid phase peptide synthesis at a purity of 95%, and qualified by HPLC and Mass Spectrometry in Polypeptide Labs (San Diego, CA). COG1410 is acetyl-AS-Aib-LRKLAib-KRLL-amide (SEQ ID NO: 1), which is derived from apoE residues 138-149 with Aib (amino isobutyric acid) substitutions at positions 140 and 145. For all experiments, peptide was dissolved in sterile saline immediately before use.

Peptide compounds SEQ ID NOs: 2-19 were synthesized similarly as described above for peptide compound SEQ ID NOs 1. Specifically, the peptide compounds were synthesized using conventional solid phase peptide synthesis at a purity of 95% and qualified by HPLC and Mass Spectrometry in Polypeptide Labs (San Diego, CA).

More specifically, solid phase synthesis of the peptide compounds SEQ ID NOs: 1-19 was performed as follows, using condensation reactions well-known to those of ordinary skill in the art. Solid-phase peptide resins were added one by one, and the condensation reaction results in the correct peptide sequence. The amino acid condensation adopts the polypeptide solid-phase synthesis (SPPS) process, and the condensation starts from the N-terminal, and the amino acid side chain is protected, and its amino group is protected with Fmoc. The general SPPS process is a repeated cycle of alternating N-terminal deprotection and condensation reactions, requiring washing of the resin between each step. After all the amino acid condensations are complete, picolinic acid is condensed into the sequence to give the full-length polypeptide bound to the support resin. Monitoring of each condensation reaction is by the applicable in-process control tests (ninhydrin test, TNBS test and/or analytical HPLC test).

The salient feature of adding an acetyl, octanyl, or picolinyl group is that each involves adding a carboxyl group to a free amino group to create an amide bond between them. This amide is typically called a peptide bond when it is used to join two amino acids together. Thus, the amide bond is the result of a dehydration condensation reaction that joins the carboxyl group to the amino group in a covalent fashion with the elimination of a water molecule.

Cleavage of the polypeptide from the support resin is under temperature-controlled conditions using trifluoroacetic acid (TFA), triisopropylsilane (TIS), 3,6-dioxin-1,8-octanedithiol (DODT) and water. After cleavage is complete, the mixture was filtered, the resin was washed, and the collected filtrate centrifuged. The peptide collected in the filtrate was precipitated with methyl tert-butyl ether (MTBE) and filtered. The resulting crude peptide solution was dried and packaged.

The crude peptide was subjected to preliminary purification and recycle purification using trifluoroacetic acid, acetonitrile, and water buffer system. According to the purity results obtained with an analytical HPLC method, the co-eluted polypeptide fractions obtained from the purification were collected, analyzed, and pooled. This pool was then subjected to further chromatography to change the counter-ion salt.

Final salt exchange (FSE) and elution were performed using preparative HPLC to convert the purified peptide to an acetate salt using a buffer system of ammonium acetate, acetonitrile (ACN) and water. This was followed by a steep gradient wash with acetic acid, ACN and water buffer to elute the peptide. Analytical method of HPLC is used to determine the purity and pooling of salt exchange components. Pooled sub-batches meeting target harvest purity criteria were lyophilized. The salt-exchanged purified peptide was lyophilized using a tray lyophilizer to remove water and remaining residual organic solvents and obtain the final peptide drug substance.

### Example 2. COG1410 has Antibacterial Activity Against PDR-A. baumannii

### MATERIALS and METHODS

### Strains and Cultures

A panel of Gram-positive and Gram-negative strains were evaluated in this study including Bacillus subtilis, vancomycin-resistant Enterococcus faecalis, E. faecium, Mycobacterium tuberculosis, M. smegmatis, Enterobacter cloacae, Escherichia coli, Citrobacter freundii, Porphyromonas gingivalis, Streptococcus pneumoniae, methicillin-sensitive Staphylococcus aureus, methicillin-resistant S. aureus (MRSA), Klebsiella pneumoniae, Pseudomonas aeruginosa, and pan-drug resistant (PDR) Acinetobacter baumannii YQ4. Strain stocks were maintained at −80° C. in 10% glycerol. Bacteria were streaked on fresh plates before each experiment. Most of them were cultured in LB broth containing 10 g/L NaCl at 37° C., except for Mycobacteria in 7H9 broth and Enterococcus in BHI broth. Porphyromonas gingivalis was cultured in BHI broth supplemented with 1 μg/ml vitamin k1, 5 g/ml hemin and 5 mg/ml L-cysteine hydrochloride at 37° C. in an anaerobic chamber. The minimal inhibitory concentrations (MICs) of antimicrobials were determined using microdilution in the corresponding broth.

The pandrug-resistant A. baumannii YQ4 was collected from a clinical laboratory and the complete genome sequence was deposited with GenBank with accession number CP053033.

### Antibacterial Activity of COG1410

For dynamic time-kill assay, the overnight culture of A. baumannii YQ4 was transferred to fresh broth with initial OD₆₀₀ 0.01 and grown to the logarithmic phase. 1 ml culture was harvested, washed twice with 1× phosphate-buffered saline (PBS, pH7.4), containing 8 mM NaH₂PO₄, 2 mM KH₂PO₄, 2.6 mM KCl, 136 mM NaCl), and finally suspended in 1 ml PBS with OD₆₀₀ 0.5, which approximately corresponds to 1×10⁸ CFU/ml. The suspension was supplemented with 16 μg/ml (1×MIC) or 80 μg/ml (5×MIC) COG1410, respectively, and incubated at 37° C. without shaking. 100 μl aliquot was respectively taken at 0, 5, 10 and 30 min, serially diluted in PBS and plated on LB agar. The CFUs were counted after incubation at 37° C. for 18 h. 16 μg/ml (1×MIC) or 80 μg/ml (5×MIC) Polymyxin B was used as positive control. Three independent experiments were performed.

Antibacterial activity in plasma of COG1410 was performed as described previously [22]. Briefly, the log-phase culture of A. baumannii YQ4 was exposed to different concentrations of COG1410 in PBS or PBS supplemented with 50% (v/v) pooled human plasma. After incubation with shaking at 200 rpm at 37° C. for 2 hours, the CFUs were counted on LB agar. LC_{99.9} indicates the lowest peptide concentration that kills ≥99.9% bacteria (ie. a 1000 fold reduction). The experiments were conducted independently for three times.

### Biofilm Inhibition and Eradication Assay

Static biofilm inhibition was performed as described previously [23]. Briefly, the log-phase culture of A. baumannii YQ4 was prepared as described above. 200 μl culture was seeded in each cell of the 96-well PVC plate with OD₆₀₀ 0.01 (ca. 1×10⁶ CFU/ml), which was exposed to different COG1410 solution with the final concentrations ranging from 0.5 to 128 μg/ml. Each concentration was determined in 8 wells. After incubation 37° C. for 48 h, the planktonic bacteria were removed by washing three times with sterilized water, followed by fixing in methanol for 15 min and staining with 0.1% crystal violet (CV) for 15 min. Excess CV was washed off and the bound CV was eluted from the biofilm into 150 μl of 95% ethanol and quantitated spectrophotometrically at 600 nm using a Biotek Synergy H1 plate reader. The experiments were performed in triplicate.

Biofilm eradication assay was performed as described previously [24]. A. baumannii YQ4 culture at log phase was diluted into fresh LB broth to OD₆₀₀ 0.01 and 200 μl per well dispensed into a 96-well PVC plate. Plates were incubated at 37° C. for 48 h and washed with PBS for three times. Serial dilutions of COG1410 (0.5-128 μg/mL) were prepared using the same media and were dispensed with 200 μl into each well. Each concentration was determined in 8 wells. LB without COG1410 was used as untreated control. After incubation for another 24 h, the remaining biofilm was quantified as described above. The experiments were performed in triplicate.

### Stability of COG1410 in Human Plasma

In order to determine the stability in human plasma, COG1410 was dissolved in 1 ml of 100% human plasma at a final concentration of 10 mg/mL and incubated at 37° C. without shaking. 100 μl aliquots were taken at 0, 1, 2, 4, 6, 8, 10 h. The log-phase culture of A. baumannii YQ4 was prepared as described above. 200 μl culture was mixed with 6 mL 0.8% soft agar to make a two-layer plate. After air dry for 30 min, four 6-mm paper disks were placed on the top and 6 μl aliquots of COG1410 at different time points were dropped on the paper disks. After 18 h incubation at 37° C., the inhibition zones were recorded by digital camera and the inhibition diameters were measured by image J. Three independent experiments were performed.

### Scanning Electron Microscopy (SEM) and Transmission Electron Microscope (TEM)

The log-phase culture of A. baumannii YQ4 was prepared as described above, harvested and washed once with PBS. The pellets were suspended in PBS supplementing with 1×COG1410 (16 μg/ml) or 1× polymyxin B (16 μg/ml) and incubated at 37° C. for 30 min. The untreated culture was used as a positive control to observe intact cells. Then, the pellets were fixed with 4% paraformaldehyde and 2.5% glutaraldehyde in 0.1 M sodium cacodylate and stored at 4° C. overnight. After wash three times with PBS, the cells were post-fixed in 1% osmium at 4° C. for 1 h. After a thorough washing in distilled water for 1 h, the bacteria were dehydrated in ascending concentrations of ethanol. Finally, these samples were transferred to Thermo fisher Quattro SEM for image collection after tert-butanol freeze-drying and sputtering coating.

The bacteria were treated with COG1410 or polymyxin B as above. Samples were then fixed in 4% paraformaldehyde and 2.5% glutaraldehyde in 0.2 M sodium cacodylate buffer (pH 7.2), post-fixed in 1% buffered osmium tetroxide, dehydrated in gradient ethanol, embedded in epoxy resin and polymerized at 60° C. for 2 days. Ultrathin sections (50-70 nm) of samples were made and placed on the copper grids, stained with uranyl acetate and lead citrate, and transferred to Thermo fisher Talos L120C TEM for image collection.

### Confocal Laser Scanning Microscopy

Cytoplasmic membrane damage was determined as described previously [25], with the Live/Dead BacLight bacterial viability kit (Invitrogen L7012). The log-phase culture of A. baumannii YQ4 was washed twice and suspended in PBS with the final OD₆₀₀ 0.1. The suspension was incubated with 1×MIC COG1410 (16 μg/ml) at 37° C. for 30 min. 5×MIC polymyxin B (40 μg/ml) and untreated cells were used as a positive control and a negative control, respectively. Then, the cells were stained with 7.5 μM SYTO-9 and 30 μM propidium iodide (PI) in dark for 15 min. 5 μl culture was spotted onto a clean slide coated with a thin layer of 1% agarose. The fluorescence was observed by Olympus FV3000 confocal laser scanning microscope. For SYTO-9, the excitation and emission were 483 nm and 503 nm, respectively. For PI, the excitation and emission were 493 nm and 636 nm, respectively.

To explore the localization of COG1410 in bacteria, the log-phase cultures of A. baumannii YQ4, K. pneumonia ATCC2146, S. aureus ATCC29213 and E. faecium 11-47 were prepared, washed and suspended in PBS with final OD₆₀₀ 0.1. These cultures were exposed to FITC-labeled COG1410 (8 μg/ml) for 30 min at room temperature, followed by co-stain with 1.6 μM membrane dye FM4-64. For each strain, 5 μl culture was spotted on a clean slide coated with a thin layer of 1% agarose. The fluorescence was observed by Olympus FV3000 confocal laser scanning microscope. For FITC, the excitation and emission were 488 nm and 525 nm, respectively. For FM4-64, the excitation and emission were 558 nm and 734 nm, respectively.

### ATP Leakage Assay

ATP leakage assay was performed using the Enhanced ATP Assay Kit of Beyotime (S0027) according to the manufacturer's manual. Briefly, the log-phase culture of A. baumannii YQ4 was prepared, washed and suspended in PBS as above. This suspension was exposed to 16 μg/ml COG1410, 16 μg/ml polymyxin B and 32 μg/ml tigecycline, respectively and incubated at 37° C. for 30 min. The supernatant was harvested and used for measurement of ATP levels. 100 μl of supernatant was mixed with 100 μl working solution and the chemiluminescence was measured by Biotek Synergy H1 plate reader. Untreated sample was set as negative control. The experiments were performed in triplicate.

### DNA Binding Assay

Gel retardation experiments were conducted as previously described [26]. Briefly, 300 ng of the plasmid pUC18 was mixed with different concentrations of COG1410 in 30 μl buffer (10 mM Tris-HCl, 1 mM EDTA buffer, pH 8.0) and incubated at room temperature for 30 min. The reaction mixtures were mixed with 1× native loading buffer and was subjected to 1.5% agarose gel electrophoresis. The migration of DNA was detected by the fluorescence of Gelred dye.

### RNA-Seq Analysis

To determine the effect of COG1410 on gene transcription, A. baumannii YQ4 was cultured in 50 ml M9 medium supplemented with 20% glucose as the sole carbon source, treated with or without 0.25×MIC of COG1410 (4 μg/ml). The experiment was conducted in triplicate. When the OD₆₀₀ reached 0.8, cells were collected by centrifuge at 4° C. and frozen in liquid nitrogen. The samples were shipped with dry ice to Guangdong Magigene Biotechnology Co., Ltd. (Guangzhou, China). Total RNA was extracted and purified using the TransZol Up Plus RNA Kit and EasyPure RNA Purification Kit, and rRNA was removed using Ribo-Zero rRNA Removal Kit, according to the manufacturer's instructions. The whole libraries for Illumina sequencing were generated by using NEB Next® Ultra™ Directional RNA Library Prep Kit. stem. After cluster generation, the library was sequenced on an Illumina Novaseq6000 platform and 150 bp paired end reads were generated. The raw data were filtered by fastp and rRNA sequences were removed [27]. Differentially expressed genes were identified using the edgeR program. Genes with the FDR 0.05 and a log 2 (fold change)>1 were taken as candidate genes. Gene Ontology (GO) and Kyoto Encyclopedia of Genes and Genomes (KEGG) enrichment analysis of differentially expressed genes were implemented by the cluster Profiler. The raw data have been deposited to SRA database with the accession number PRJNA833738.

### ROS Detection

The intracellular ROS level was determined by Reactive Oxygen Species Assay Kit of Beyotime Biotechnology (S0033S), according to the manufacturer's instruction. The log-phase culture of A. baumannii YQ4 was harvested and washed in PBS, and then diluted 10 fold to approximately 107 CFU/mL. 1 μl DCFH-DA (10 μM) was added to 1 ml cell culture and incubated at 37° C. for 20 min. Then the fluorescence probe was fully removed by PBS wash for three times and resuspended in PBS, followed by addition of COG1410 (16 μg/ml) or water. Rosup (50 μg/ml) was positive control in the kit. The culture was incubated at 37° C. for 30 min. The fluorescence intensity was measured by plate reader at excitation of 488 nm and emission of 525 nm. The experiment was performed in duplicate.

### Evaluation of Induction of Resistance Through Serial Passage

To evaluate the drug resistance barrier of COG1410, A. baumanii YQ4 were cultured in LB broth with constant shaking at 150 rpm at 37° C., with exposure to sub-MIC levels of COG1410 or polymyxin B. 20 μl of culture was transferred into 2 ml fresh medium every day. The initial concentration of the tested compounds was set up as 1/32×MIC and doubled every 10 passages. The 1 ml of bacterial culture was stored in 10% sterile glycerol at −80° C. every 5 passages. The MIC value of collected cultures and original strains were measured through the micro-dilution method.

### Hemolysis Assay and Cytotoxicity Assay

The human red blood cell (RBC) hemolytic activity of COG1410 was measured according to the protocol described previously with minor modification [28]. The anti-coagulated (citrate) whole blood was pelleted by centrifugation at 700 g for 8 min, washed three times with PBS, and suspended to 0.5% (vol/vol) in PBS. 75 μl RBC suspension was transferred to each well of V-bottom 96 well plate, where an equal volume of COG1410 was prepared with 2-fold dilutions in PBS. The highest concentration was 512 μg/ml (363 μM). PBS and Triton X-100 (0.1%) were used as negative and positive controls, respectively. The plate was incubated at 37° C. for 1 h, followed by centrifuge at 1,000 rpm for 5 min at 4° C. 60 μl aliquots of the supernatant from each well were quickly transferred to a new flat-bottom 96-well plate. The optical absorbance at OD₄₁₄ was measured with a microplate reader (BioTek, synergy H1). The hemolysis percentage was then normalized with respect to the averaged negative (0%) and positive (100%) controls. Three independent experiments were conducted.

The cytotoxicity of COG1410 on normal liver cell L02 was assessed by Cell Counting Kit 8 (Solarbio, CA1210) according to the manufacturer's manual. Briefly, 100 μl human hepatic L02 cells were seeded in the 96-well plate with 4×10³ cells per well in RPMI-1640 medium containing 20% FBS, and incubated at 37° C. in a 5% CO2 atmosphere for 24 h. The cells without exposure to peptide were used as negative control. Then, the L02 cells were incubated with different concentrations of COG1410 for another 24 h. 10 μl CCK8 solution was added to each well. After 2 h incubation, the optical absorbance at 450 nm was measured with BioTek synergy H1 plate reader. The cell activity was expressed as percentage of mean absorbance divided by cells exposed to peptide with respect to results with incubation with the control. The experiments were done in triplicate.

Synergy with Antimicrobials

The checkerboard method was performed to determine the fractional inhibitory concentration (FIC) index between COG1410 and other antimicrobials. Two-fold serial dilutions of antimicrobials and COG1410 were conducted and mixed in the 96-well plate. The log-phase culture of A. baumannii YQ4 was added to each well with initial OD₆₀₀ 0.01. The plate was incubated at 37° C. for 20 h and the FIC values were calculated based on the equation: FIC=MIC (COG1410 in combination)/MIC (COG1410 alone)+MIC (antimicrobial in combination)/MIC (antimicrobial alone). The experiments were conducted twice independently.

### Nematode Killing Assay

The Caenorhabditis elegans wild-type strain Bristol N2 was used in this assay. The relative experimental manipulation was according to the previously described protocols [29]. C. elegans were grown on nematode growth medium (NGM) with E. coli OP50 lawn as a food source at 20° C. For synchronization, C. elegans eggs were harvested and hatched to stage L1 in M9 medium at 20° C., then transferred to E. coli lawns to grow to stage L4. For the in vivo killing assay, the synchronized L4 nematodes were harvested from a few NGM plates and transferred to 15 ml M9 medium containing 20% LB, 1×10⁹ log-phase cells of A. baumannii YQ4 and 10 μM FeCl₃. The infection model was established at 20° C. for 24 h. E. coli OP50 was used as negative control. The pre-infected nematodes were washed twice with M9 medium and dispensed to a 60-mm NGM agar plate with 30 nematodes each, where different concentrations of COG1410 and/or polymyxin B were supplemented, as well as 2 mM 5-Fluoro-2′-deoxyuridine-Floxuridine (FUDR). The uninfected nematodes were used as positive control for worm lifespan. Live and dead or missing nematodes were counted and recorded through a stereomicroscope every 24 h for 16 days. The survival curve of C. elegans were analyzed by Kaplan-Meier using GraphPad Prism 9. The in vivo killing assays were performed in triplicate.

### Results

### COG1410 Possesses Broad Spectrum of Antimicrobial Activity

COG1410 is a synthetic cationic peptide with a simple alpha helix, which is composed of 12 amino acids, acetyl-AS-Aib-LRKL-Aib-KRLL-amide (SEQ ID NO: 1), including 4 positively charged, 5 nonpolar and 1 polar amino acids, as well as two unnatural amino acids, Aib. The antimicrobial activity of COG1410 was determined by measuring the minimal inhibition concentration (MIC) values on a panel of Gram-positive and Gram-negative strains. In the case of Gram-positive, COG1410 inhibited the growth of Bacillus subtilis, vancomycin-resistant Enterococcus faecalis and E. faecium, Mycobacterium tuberculosis and M. smegmatis with MICs ranging from 1 to 32 μg/ml. As for Gram-negative bacteria, COG1410 showed antimicrobial activity for Enterobacter cloacae, Escherichia coli, Citrobacter freundii and even anaerobe, Porphyromonas gingivalis with MICs from 16 to 64 μg/ml. In contrast, COG1410 was inactive against Streptococcus pneumoniae, methicillin-sensitive Staphylococcus aureus, methicillin-resistant S. aureus (MRSA), Klebsiella pneumoniae and Pseudomonas aeruginosa. Remarkably, COG1410 could kill the pan-drug resistant (PDR) Acinetobacter baumannii YQ4, with MIC and minimal bactericidal concentration (MBC) 16 μg/ml (11.3 μM). The other 107 clinically collected A. baumannii strains were tested and it was found that the MICs of COG1410 ranged from 16 to 32 μg/ml.

COG1410 Shows Potent and Quick Bactericidal Efficacy Against PDR A. baumannii In Vitro

The in vitro time-kill kinetics of COG1410 against the PDR A. baumannii YQ4 was analyzed. 1×MIC COG1410 decreased almost the CFU by 3 logs (ie. 1000 fold) within 5 min in 20 μM phosphate-buffered saline (PBS) (FIG. 1A). When the concentration of AMP increased to 5×MIC, the inoculum (1×10⁸ CFU/ml) was completely eliminated within 5 min. Within 30 min, 1×MIC COG1410 killed bacteria with a 6 log reduction in CFU. In comparison, 1× and 5×MIC polymyxin B decreased CFU by 2 logs and 4 logs after 30 min, respectively (FIG. 1A). These data showed that COG1410 acted more rapidly than polymyxin B.

To determine the efficacy of COG1410 in the pooled human plasma, its LC₉₉₉ value (killing 99.9% within 2 h of incubation) was measured in PBS with or without 50% human plasma. As shown in FIG. 1B, 2 μg/ml (1.4 μM) COG1410 achieved 99.9% killing in PBS. Consistently, the antimicrobial activity of this AMP was reduced in the presence of human plasma, with LC_{99.9} up to 8 μg/ml (5.6 μM).

To address the activity of COG1410 in other conditions, the bactericidal efficacy of COG1410 in PBS, LB broth and saline, respectively, was determined. Compared with near elimination in PBS, 1×MIC COG1410 only killed 78.6% and 78.3% A. baumannii in LB broth (250 mM NaCl) and saline (225 mM NaCl) after incubation of 2 h, respectively (FIG. 1C). These data suggested that high salt might disturb the bactericidal effect of COG1410.

### COG1410 Maintains Stability in Human Plasma

In order to determine the stability of COG1410 in human plasma, 10 mg/ml COG1410 was incubated with 100% human plasma at 37° C. Samples were retrieved at different time points and dropped onto paper disks. The antimicrobial activity was evaluated by measuring the diameter size of the inhibition zone. Interestingly, the inhibition diameters of COG1410 did not significantly change within the first 2 h (data not shown). At 4 h, the activity was reduced by 6.8%. After 10 h of incubation, 20% of activity was lost. These data suggested that COG1410 was very stable in human plasma.

### COG1410 Exhibits Biofilm Inhibition and Eradication Activity

Bacterial biofilm formation is often associated with chronic wound infections and drug resistance]. To investigate the efficacy of COG1410 against biofilms, the biofilm mass formed by A. baumannii YQ4 was measured by crystal violet staining. COG1410 inhibited biofilm formation in a dose-dependent manner (FIG. 2A). 0.5×MIC of COG1410 significantly reduced biofilm formation.

1×MIC COG1410 Reduced 55% (?46%?) Biofilm Mass, Compared with Untreated Controls.

For preformed biofilms, 1×MIC of COG1410 dispersed ca. 46% of mature biofilm. If the AMP concentration increased to 4×MIC, more than 88% of biofilm was eradicated (FIG. 2B). Taken together, the data show COG1410 can be used as an anti-biofilm agent.

### COG1410 Treatment Increases Bacterial Cell Membrane Permeability

To assess the mechanism of action of COG1410, the cell morphology was first examined by scanning electron microscopy (SEM). As illustrated in FIG. 3A, untreated A. baumannii cells looked like peanuts with small spike-like patterns on their surfaces. As controls, polymyxin B treatment led to cell lysis and release of cell contents. In contrast, the majority of COG1410 treated cells were intact, with few cell debris observed. Pores were not detected on the cell surfaces. These data indicated that COG1410 might not cause pore formation or cell lysis.

In order to determine the integrity of the bacterial cell membrane, the same batch of treated cells was observed by transmission electron microscopy (TEM). The untreated cells had intact cytoplasmic membranes. Exposing to polymyxin B gradually caused a collapse of the cell walls in most cells (FIG. 3B). However, the COG1410-treated cells became wrinkled and smaller compared with untreated cells. Additionally, it seemed that the inner membrane was separated from the cell wall.

To verify the permeabilization of the plasma membrane by COG1410, it was decided to stain COG1410-treated A. baumannii YQ4 cells by two fluorescent nucleic acid dyes, SYTO-9 and propidium iodide (PI). The former could stain both live and dead cells and show green fluorescence (shown as shaded shapes in FIG. 4), but the latter only enters non-living cells and emits red fluorescence (shown as open shapes in FIG. 4). As expected, untreated cells showed green fluorescence indicating all of them were living. Both COG1410 and polymyxin B treated cells showed red fluorescence, which supports that the cells were dead (data not shown). It's worth noting that there is no green fluorescence for these treated dead cells stained by SYTO-9, which might be due to competition of PI. Thus, this staining assay confirmed that COG1410 permeabilized bacterial membranes.

Membrane disruptions can be further characterized by measuring leakage of intracellular components of bacterial cells. To address the mechanism of action of COG1410, the extracellular ATP concentration of cells exposed to 1×MIC COG1410 by Enhanced ATP Assay Kit (Beyotime) was measured. Tigecycline binds to the bacterial 30S ribosome, blocking the entry of transfer RNA, which was used as a negative control. As expected, tigecycline treated cells were similar to untreated cells with regard to ATP leakage levels. Cells exposed to Polymyxin B released more ATP compared to untreated cells. Remarkably, COG1410 treatment lead to ATP leakage, which was significantly more than untreated controls and the tigecycline treatment group (FIG. 3C). Taken together, the cationic AMP COG1410 disrupted bacterial cell membranes.

### COG1410 is Localized in the Cytoplasm

Besides direct membrane disruption, some sub-MIC AMPs may have cytoplasmic targets. To address if COG1410 directly binds with the bacterial membrane or enters the cytosol, a few bacterial pathogens were treated with 8 μg/ml COG1410 for 30 min and then co-stained with red fluorescent membrane dye. COG1410 not only entered the cytoplasms of A. baumannii and E. faecium, but also got into K. pneumoniae and S. aureus (FIG. 4). COG1410 was active against the former two strains, but did not kill the latter two strains. These results suggested that COG1410 may bind with the cell membrane of A. baumannii more easily than other bacteria or COG1410 specifically inhibited a cytoplasmic target in A. baumannii.

COG1410 Nonspecifically Binds with DNA

To address whether the cationic AMP binds with DNA, a gel retardation experiment was performed. The electrophoretic mobility of plasmid pUC18 was measured after incubation with different concentrations of COG1410. Without AMP, the plasmid normally migrated into the gel. In the presence of 1×MIC COG1410, some DNA remained in the loading well and only a fraction of the DNA migrated into the gel. With higher concentrations of COG1410, DNA mobility was completely retarded (data not shown). Taken together, COG1410 was able to bind with DNA nonspecifically in a concentration dependent manner.

### Sub-MIC of COG1410 Treatment Induces Expression of Genes Involved in Oxidation-Reduction Process

To further identify the putative intercellular targets of COG1410, RNA-seq was used to compare the transcriptome of A. baumanii YQ4 in the presence or absence of 0.25×MIC of COG1410 (4 μg/ml). To reduce interference from rich medium, M9 minimal medium was chosen to prepare bacterial cultures. The analysis of RNA-seq data identified 92 significantly differentially expressed genes (DEGs) with at least a 2-fold change. Compared with untreated controls, the transcription level of 55 and 37 genes increased and decreased in the presence of AMP, respectively. The 92 DEGs were classified into 12 categories at the GO level 2, such as catalytic activity, cellular anatomical entity, metabolic process, and response to stimulus (data not shown). Interestingly, the genes involved in oxidation-reduction processes were enriched (FIG. 5). This raised a question of whether treatment with COG1410 increased reactive oxygen species. To address this concern, the intracellular ROS level was measured using DCFH-DA probe. As shown in FIG. 3D, treatment of COG1410 significantly increased ROS production, which indicated that ROS might be another killing mechanism of COG1410.

### COG1410 is Highly Refractory to Induced Resistance

For a new antimicrobial compound, its drug resistance barrier is a pivotal parameter since the bacterial population always tries to develop resistance to survive. Especially, A. baumannii is naturally competent to adsorb exogenous DNA to acquire resistance against different kinds of antibiotics. To evaluate the rate of resistance development of A. baumannii to COG1410, the PDR strain, YQ4, was serially passaged in the presence of sub-MIC concentrations of COG1410. Polymyxin B was included as an antibiotic control. The MIC value of polymyxin B increased 64 fold after 55 passages (data not shown). In contrast, the MIC of COG1410 only increased 4 fold in YQ4 after 55 passages. These data indicated that A. baumannii may not easily disable COG1410's toxicity via genetic mutations.

### COG1410 Shows Low Hemolytic Activity and Medium Eukaryotic Cell Toxicity

The hemolytic potential of COG1410 was determined by exposing human erythrocytes to different concentrations of COG1410 and measuring the release of hemoglobin. Controls consisted of 100% release of hemoglobin following treatment with 0.1% Triton X-100 and 0% release following treatment with PBS. 128 μg/ml (8×MIC) of COG1410 lead to less than 5% hemolysis (FIG. 6A). The minimal concentration whereby half of the red blood cells are lysed (EC₅₀) was 441 μg/ml. Therefore, the selectivity index, SI (EC₅₀/MIC), was 27.5 for PDR A. baumannii YQ4.

The cytotoxic effect of COG1410 on normal human hepatic L02 cells was evaluated by CCK8 assay. As shown in FIG. 6B, there was 7% toxicity to the L02 cell at 16 μg/ml. The half-maximal (EC₅₀) toxicity concentration of COG1410 to L02 cells was 58.9 μg/ml.

The Bactericidal Action of COG1410 was Different from Polymyxin B

To address whether the antimicrobial effect of COG1410 depends on negatively charged lipopolysaccharide (LPS) on the cell surface, the susceptibility was compared of A. baumannii ATCC19606 and two LPS-modified strains, pmrA^{P102R} and pmrA^{P102R}miaA^{I221V} [34]. pmrA mutation leads to derepression of PmrC, which encodes lipid A phosphoethanolamine transferase [35]. It was found that lipidA modification of LPS did not change the antimicrobial efficacy of COG1410. This was in contrast to polymyxin B, the last resort for MDR Gram-negative bacteria, whose bactericidal effect depends on the electrostatic interaction with LPS (FIG. 7A). These data suggest that COG1410 may have a different antibacterial mechanism from polymyxin B and there is no cross-resistance between them.

COG1410 Shows Strong Synergy with Polymyxin B

To investigate whether COG1410 has synergistic interactions with conventional antibiotics, the synergy was measured using COG1410 in combination with a few frequently-used antibiotics against A. baumanii in medical practice. As shown in FIG. 7A-7C, COG1410 displayed synergy with polymyxin B, Ceftazidime and tetracycline with FIC values of 0.13, 0.31 and 0.31, respectively. Surprisingly, only 2 μg/ml COG1410 plus 1 μg/ml polymyxin B were able to completely inhibit growth of the PDR strain (FIG. 7B). Time-kill kinetics showed that this combination displayed similar bactericidal activity to 16 μg/ml COG1410 alone (FIG. 7C).

The Combined Therapy of COG1410 and Polymyxin B is Capable of Rescuing C. elegans Infected by A. Baumannii

To address the effectiveness in vivo, C. elegans was utilized to develop a model of bacterial infection. L4 nematodes were pre-infected in M9 buffer and transferred to NGM agar plate supplemented with or without COG1410. To increase the pathogenesis of A. baumannii, 10 μM FeCl₃ was added as has been previously reported [36]. FIG. 8 is a graph illustrating that the combined therapy of COG1410 and polymyxin B rescued infected nematodes. C. elegans were pre-infected by A. baumannii YQ4 and transferred to a NGM plate supplemented with 16 μg/ml COG1410 or 2 μg/ml COG1410 and 1 μg/ml polymyxin B. The dead nematodes were counted every day for 2 weeks. The survival curve was analyzed by Kaplan-Meier method and the statistical significance was analyzed by Log-rank test. **, p<0.01. Compared with the untreated group, 16 μg/ml COG1410 alleviated the death of infected nematodes, but the change was not statistically significantly. Unexpectedly, the combination therapy of COG1410 (2 μg/ml) plus polymyxin B (1 μg/ml) significantly rescued the pre-infected nematodes and even elongated their lives.

### Example 3. Antibacterial Activity of Peptide Compounds Against Bacterial Strains Including P. gingivalis

Minimum inhibitory concentrations (MIC) were measured for peptide compounds shown in Table 1 herein above. Briefly, P. gingivalis is grown under anaerobic conditions to mid-log phase as per Dominy et al. (2019) in anaerobic BHI broth (Brain-Heart-Infusion broth with 0.5 mg/ml L-cysteine, 0.5 mg/ml arginine, 5 μg/ml hemin and 1.0 μg/ml Vitamin K) at 37° C. for 24 to 96 hours. L. salivarius is grown under 5% CO₂ conditions in BHI broth at 37° C. for 24 to 48 hours. The microtiter plate method outlined by Jansen (ibid) was used to measure MICs. Compounds and bacteria are added to wells of a microtiter plate and grown anaerobically (P. gingivalis) or with CO₂ (L. salivarius) for 24 to 72 hours. Growth is measured with light scattering at 592 nm on a plate reader and absorbance plotted against μg/ml of drug in each well. The μg/ml of each compound at which the absorbance does not significantly increase over background (media alone) by the endpoint of the time course is the MIC.

One skilled in the art will readily appreciate that the present disclosure is well adapted to carry out the objects and obtain the ends and advantages mentioned, as well as those inherent therein. The present disclosure is representative of embodiments, which are exemplary, and are not intended as limitations on the scope of the present disclosure. Changes and other uses will occur to those skilled in the art which are encompassed within the spirit of the present disclosure as defined by the scope of the claims.

No admission is made that any reference, including any non-patent or patent document cited in this specification, constitutes prior art. It will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

### REFERENCES

1. Alsan, M. and M. Klompas, Acinetobacter baumannii: An Emerging and Important Pathogen. Journal of clinical outcomes management: JCOM, 2010. 17(8): p. 363-369.
2. Boucher, H. W., et al., Bad bugs, no drugs: no ESKAPE! An update from the Infectious Diseases Society of America. Clin Infect Dis, 2009. 48(1): p. 1-12.
3. Lima, W. G., et al., Chromosomally encoded and plasmid-mediated polymyxins resistance in Acinetobacter baumannii: a huge public health threat. Eur J Clin Microbiol Infect Dis, 2018. 37(6): p. 1009-1019.
4. Hernández-González, I. L. and S. Castillo-Ramírez, Antibiotic-resistant <em>Acinetobacter baumannii</em> is a One Health problem. The Lancet Microbe, 2020. 1(7): p. e279.
5. Perez, F., et al., Global Challenge of Multidrug-Resistant <i>Acinetobacter baumannii</i>. Antimicrobial Agents and Chemotherapy, 2007. 51(10): p. 3471-3484.
6. Mahlapuu, M., et al., Antimicrobial Peptides: An Emerging Category of Therapeutic Agents. Frontiers in Cellular and Infection Microbiology, 2016. 6(194).
7. Zhang, L.-j. and R. L. Gallo, Antimicrobial peptides. Current Biology, 2016. 26(1): p. R14-R19.
8. Groß, A., et al., Synthetic Peptides as Protein Mimics. Frontiers in Bioengineering and Biotechnology, 2016. 3(211).
9. Liu, C.-C., et al., Apolipoprotein E and Alzheimer disease: risk, mechanisms and therapy. Nature reviews. Neurology, 2013. 9(2): p. 106-118.
10. Tensaouti, Y., T.-S. Yu, and S. G. Kernie, Apolipoprotein E regulates the maturation of injury-induced adult-born hippocampal neurons following traumatic brain injury. PLOS ONE, 2020. 15(3): p. e0229240.
11. Wang, C. Q., et al., An apolipoprotein E mimetic peptide with activities against multidrug-resistant bacteria and immunomodulatory effects. J Pept Sci, 2013. 19(12): p. 745-50.
12. Laskowitz, D. T., et al., COG1410, a novel apolipoprotein E-based peptide, improves functional recovery in a murine model of traumatic brain injury. J Neurotrauma, 2007. 24(7): p. 1093-107.
13. Pane, K., et al., A new cryptic cationic antimicrobial peptide from human apolipoprotein E with antibacterial activity and immunomodulatory effects on human cells. The FEBS Journal, 2016. 283(11): p. 2115-2131.
14. Kelly, B. A., et al., ApolipoproteinE-derived antimicrobial peptide analogues with altered membrane affinity and increased potency and breadth of activity. The FEBS Journal, 2007. 274(17): p. 4511-4525.
15. Azuma, M., et al., A synthetic peptide of human apoprotein E with antibacterial activity. Peptides, 2000. 21(3): p. 327-330.
16. Laskowitz, D. T., et al., The apoE-mimetic peptide, COG1410, improves functional recovery in a murine model of intracerebral hemorrhage. Neurocrit Care, 2012. 16(2): p. 316-26.
17. Tukhovskaya, E. A., et al., COG1410, a novel apolipoprotein-E mimetic, improves functional and morphological recovery in a rat model of focal brain ischemia. Journal of Neuroscience Research, 2009. 87(3): p. 677-682.
18. Kuai, L., et al., Apolipoprotein E-Mimetic Peptide COG1410 Enhances Retinal Ganglion Cell Survival by Attenuating Inflammation and Apoptosis Following TONI. Frontiers in Neuroscience, 2019. 13(980).
19. Wu, Y., et al., An apoE-derived mimic peptide, COG1410, alleviates early brain injury via reducing apoptosis and neuroinflammation in a mouse model of subarachnoid hemorrhage. Neuroscience Letters, 2016. 627: p. 92-99.
20. Zhang, S.-S., et al., A combination of receptor mediated transcytosis and photothermal effect promotes BBB permeability and the treatment of meningitis using itraconazole. Nanoscale, 2020. 12(46): p. 23709-23720.
21. Zhang, S., et al., Multifunctional nanorods based on self-assembly of biomimetic apolipoprotein E peptide for the treatment of Alzheimer's disease. J Control Release, 2021. 335: p. 637-649.
22. de Breij, A., et al., The antimicrobial peptide SAAP-148 combats drug-resistant bacteria and biofilms. Science Translational Medicine, 2018. 10(423): p. caan4044.
23. Gaddy, J. A. and L. A. Actis, Regulation of Acinetobacter baumannii biofilm formation. Future microbiology, 2009. 4(3): p. 273-278.
24. Mwangi, J., et al., The antimicrobial peptide ZY4 combats multidrug-resistant <em>Pseudomonas aeruginosa</em> and <em>Acinetobacter baumannii</em> infection. Proceedings of the National Academy of Sciences, 2019. 116(52): p. 26516-26522.
25. Farkas, A., et al., Comparative Analysis of the Bacterial Membrane Disruption Effect of Two Natural Plant Antimicrobial Peptides. Frontiers in Microbiology, 2017. 8(51).
26. Yan, J., et al., Two hits are better than one: membrane-active and DNA binding-related double-action mechanism of NK-18, a novel antimicrobial peptide derived from mammalian NK-lysin. Antimicrob Agents Chemother, 2013. 57(1): p. 220-8.
27. Chen, S., et al., fastp: an ultra-fast all-in-one FASTQ preprocessor. Bioinformatics, 2018. 34(17): p. i884-1890.
28. Oddo, A. and P. R. Hansen, Hemolytic Activity of Antimicrobial Peptides. Methods Mol Biol, 2017. 1548: p. 427-435.
29. Stiernagle, T., Maintenance of C. elegans. WormBook, 2006: p. 1-11.
30. He, X., et al., Biofilm Formation Caused by Clinical Acinetobacter baumannii Isolates Is Associated with Overexpression of the AdeFGH Efflux Pump. Antimicrobial Agents and Chemotherapy, 2015. 59(8): p. 4817-4825.
31. Greer, N. D., Tigecycline (Tygacil): the first in the glycylcycline class of antibiotics. Proceedings (Baylor University. Medical Center), 2006. 19(2): p. 155-161.
32. Vasilchenko, A. S. and E. A. Rogozhin, Sub-inhibitory Effects of Antimicrobial Peptides. Frontiers in Microbiology, 2019. 10(1160).
33. Domingues, S., et al., Competence for Natural Transformation Is Common among Clinical Strains of Resistant Acinetobacter spp. Microorganisms, 2019. 7(2): p. 30.
34. Sun, B., et al., New Mutations Involved in Colistin Resistance in <span class=“named-content genus-species” id=“named-content-1”>Acinetobacter baumannii</span>. mSphere, 2020. 5(2): p. e00895-19.
35. Arroyo, L. A., et al., The pmrCAB operon mediates polymyxin resistance in Acinetobacter baumannii ATCC 17978 and clinical isolates through phosphoethanolamine modification of lipid A. Antimicrob Agents Chemother, 2011. 55(8): p. 3743-51.
36. Vallejo, J. A., et al., Optimisation of the Caenorhabditis elegans model for studying the pathogenesis of opportunistic Acinetobacter baumannii. Int J Antimicrob Agents, 2015.
37. Colquhoun, J. M. and P. N. Rather, Insights Into Mechanisms of Biofilm Formation in Acinetobacter baumannii and Implications for Uropathogenesis. Frontiers in Cellular and Infection Microbiology, 2020. 10(253).
38. Dijksteel, G. S., et al., Review: Lessons Learned From Clinical Trials Using Antimicrobial Peptides (AMPs). Frontiers in Microbiology, 2021. 12(287).
39. Lu, J., et al., D- and Unnatural Amino Acid Substituted Antimicrobial Peptides With Improved Proteolytic Resistance and Their Proteolytic Degradation Characteristics. Frontiers in Microbiology, 2020. 11(2869).
40. Peng, J., et al., Antibacterial mechanism of peptide Cec4 against Acinetobacter baumannii. Infect Drug Resist, 2019. 12: p. 2417-2428.
41. Choi, H., Z. Yang, and J. C. Weisshaar, Oxidative stress induced in E. coli by the human antimicrobial peptide LL-37. PLOS Pathog, 2017. 13(6): p. e1006481.
42. Sochacki, K. A., et al., Real-time attack on single <i>Escherichia coli</i> cells by the human antimicrobial peptide LL-37. Proceedings of the National Academy of Sciences, 2011. 108(16): p. E77-E81.
43. Abdul Rahim, N., et al., Synergistic killing of NDM-producing MDR Klebsiella pneumoniae by two ‘old’ antibiotics-polymyxin B and chloramphenicol. The Journal of antimicrobial chemotherapy, 2015. 70(9): p. 2589-2597.
44. Ruden, S., et al., Synergy Pattern of Short Cationic Antimicrobial Peptides Against Multidrug-Resistant Pseudomonas aeruginosa. Frontiers in Microbiology, 2019. 10.
45. Ho, M. H., R. J. Lamont and H. Xie (2017). “Identification of Streptococcus cristatus peptides that repress expression of virulence genes in Porphyromonas gingivalis.” Sci Rep 7(1): 1413. Access. No's.: 28469253 PMC5431200.
46. Ho, M. H., R. J. Lamont and H. Xie (2017). “A novel peptidic inhibitor derived from Streptococcus cristatus ArcA attenuates virulence potential of Porphyromonas gingivalis.” Sci Rep 7(1): 16217. Access. No's.: 29176569 PMC5701168.
47. Dominy, S. S., C. Lynch, F. Ermini, M. Benedyk, A. Marczyk, A. Konradi, M. Nguyen, U. Haditsch, D. Raha, C. Griffin, L. J. Holsinger, S. Arastu-Kapur, S. Kaba, A. Lee, M. I. Ryder, B. Potempa, P. Mydel, A. Hellvard, K. Adamowicz, H. Hasturk, G. D. Walker, E. C. Reynolds, R. L. M. Faull, M. A. Curtis, M. Dragunow and J. Potempa (2019). “Porphyromonas gingivalis in Alzheimer's disease brains: Evidence for disease causation and treatment with small-molecule inhibitors.” Sci Adv 5(1): eaau3333. Access. No's.: 30746447 PMC6357742.

1. A method for inhibiting microbial growth, comprising: contacting a microbe with an effective amount of a peptide compound to inhibit the microbial growth, wherein the peptide compound comprises:
(COG1410) acetyl-AS-aib-LRKL-aib-KRLL-amide (SEQ ID NO: 1);
Picolinyl-AS-aib-LRKL-aib-KRLL-amide (SEQ ID NO: 2);
Picolinyl-AS-C-LRKL-aib-KRLL-C-amide (SEQ ID NO: 3); wherein there is a disulfide link between the two cysteine residues;
Acetyl-LLRK-aib-LKRL-aib-SA-CONH2 (SEQ ID NO: 4);
Acetyl-llrk-Aib-lkkl-Aib-sa-amide (SEQ ID NO: 5), wherein all the amino acid residues are D-amino acids;
Acetyl-as-aib-lrkl-aib-krll-amide (SEQ ID NO: 6), wherein all the amino acid residues are D-amino acids;
Acetyl-LLRK-aib-LRKL-aib-SAS-aib-LRKL-aib-KRLL-CONH2 (SEQ ID NO: 7);
Acetyl-LRVRCAS-aib-LRKL-aib-KRLL-CONH2 (SEQ ID NO: 8);
Acetyl-LRVRLAS-aib-LKKL-aib-KRLL-Amide (SEQ ID NO: 9);
Acetyl-LRVRLAS-aib-LRKL-aib-KRLL-Amide (SEQ ID NO: 10);
Acetyl-llrk-aib-lkrl-aib-salrvrl-amide (SEQ ID NO: 11), wherein all the amino acid residues are D-amino acids;
Acetyl-LRVRLASHLRKLRKRLLAS-aib-LRKL-aib-KRLL-CONH2 (SEQ ID NO: 12);
C8-AS-aib-LRKL-aib-KRLL-amide (SEQ ID NO: 13);
Acetyl-K(C8)-AS-aib-LRKL-aib-KRLL-amide (SEQ ID NO: 14);
Acetyl-K(Picolinyl)-AS-aib-LRKL-aib-KRLL-amide (SEQ ID NO: 15);
Acetyl-LRVRLASHLRKLRKRLLR-amide (SEQ ID NO: 16);
Acetyl-LRKLRKRLLLRKLRKRLL-amide (SEQ ID NO: 17);
Acetyl-LRVRLASHLRKLRKRLLRDADDLQKRLAVY-amide (SEQ ID NO: 18); or
Picolinyl-llrk-aib-lkrl-aib-salrvrl-amine (SEQ ID NO: 19), wherein all the amino acid residues are D-amino acids,
wherein aib is amino isobutyric acid.

2. The method of claim 1, wherein the peptide compound comprises SEQ ID NO: 1, SEQ ID NO: 2, SEQ ID NO: 3, SEQ ID NO: 13, SEQ ID NO: 14, or SEQ ID NO: 15.

3. The method of claim 1, wherein the peptide compound comprises SEQ ID NO: 1, SEQ ID NO: 2, or SEQ ID NO: 3.

4. The method of claim 1, wherein the peptide compound comprises SEQ ID NO: 1.

5. The method of claim 1, wherein the microbe comprises a bacterium that is one or more of multidrug-resistant (MDR), extensively drug-resistant (XDR), or pandrug-resistant (PDR).

6. The method of claim 1, wherein the microbe comprises Porphoryomas gingivalis.

7. The method of claim 1, wherein the microbe comprises a Gram-negative bacterial pathogen.

8-15. (canceled)

16. A method of treating a subject having a microbial infection, the method comprising administering to a subject a therapeutically effective amount of a pharmaceutical composition comprising a peptide compound, or a pharmaceutically acceptable salt or solvate thereof, to inhibit microbial growth in the subject, wherein the peptide compound comprises:
(COG1410) acetyl-AS-aib-LRKL-aib-KRLL-amide (SEQ ID NO: 1);
Picolinyl-AS-aib-LRKL-aib-KRLL-amide (SEQ ID NO: 2);
Picolinyl-AS-C-LRKL-aib-KRLL-C-amide (SEQ ID NO: 3); wherein there is a disulfide link between the two cysteine residues;
Acetyl-LLRK-aib-LKRL-aib-SA-CONH2 (SEQ ID NO: 4);
Acetyl-llrk-Aib-lkkl-Aib-sa-amide (SEQ ID NO: 5), wherein all the amino acid residues are D-amino acids;
Acetyl-as-aib-lrkl-aib-krll-amide (SEQ ID NO: 6), wherein all the amino acid residues are D-amino acids;
Acetyl-LLRK-aib-LRKL-aib-SAS-aib-LRKL-aib-KRLL-CONH2 (SEQ ID NO: 7);
Acetyl-LRVRCAS-aib-LRKL-aib-KRLL-CONH2 (SEQ ID NO: 8);
Acetyl-LRVRLAS-aib-LKKL-aib-KRLL-Amide (SEQ ID NO: 9);
Acetyl-LRVRLAS-aib-LRKL-aib-KRLL-Amide (SEQ ID NO: 10);
Acetyl-llrk-aib-lkrl-aib-salrvrl-amide (SEQ ID NO: 11), wherein all the amino acid residues are D-amino acids;
Acetyl-LRVRLASHLRKLRKRLLAS-aib-LRKL-aib-KRLL-CONH2 (SEQ ID NO: 12);
C8-AS-aib-LRKL-aib-KRLL-amide (SEQ ID NO: 13);
Acetyl-K(C8)-AS-aib-LRKL-aib-KRLL-amide (SEQ ID NO: 14);
Acetyl-K(Picolinyl)-AS-aib-LRKL-aib-KRLL-amide (SEQ ID NO: 15);
Acetyl-LRVRLASHLRKLRKRLLR-amide (SEQ ID NO: 16);
Acetyl-LRKLRKRLLLRKLRKRLL-amide (SEQ ID NO: 17);
Acetyl-LRVRLASHLRKLRKRLLRDADDLQKRLAVY-amide (SEQ ID NO: 18); or
Picolinyl-llrk-aib-lkrl-aib-salrvrl-amine (SEQ ID NO: 19), wherein all amino acid residues are D-amino acids,
wherein aib is amino isobutyric acid.

17. The method of claim 16, wherein the microbial growth is caused by a bacterium comprising one or more of multidrug-resistant (MDR), extensively drug-resistant (XDR), or pandrug-resistant (PDR).

18. The method of claim 16, wherein the microbial growth is caused by an oral infection with a bacterium comprising Porphoryomas gingivalis.

19. The method of claim 16, wherein the microbial growth is caused by a Gram-negative bacterial pathogen.

20. (canceled)

21. The method of claim 16, wherein the microbial growth is caused by a Gram-positive pathogen.

22. (canceled)

23. The method of claim 16, wherein the administering is in combination with one or more antimicrobial compounds.

24-28. (canceled)

29. The method of claim 16, wherein the administering comprises a single administration or multiple administrations of the composition comprising the peptide compound.

30. The method of claim 16, wherein the composition comprising the peptide compound is administered topically, enterally, systemically, or parenterally.

31. The method of claim 16, wherein the microbial growth is caused by an oral infection with a bacterium comprising Porphoryomas gingivalis and the composition comprising the peptide compound is administered in a formulation of a mouthwash or a toothpaste.

32. The method of claim 16, wherein the subject is a mammal, a primate, or a human.

33. An antimicrobial composition comprising:
a therapeutically effective amount of a peptide compound, or a pharmaceutically acceptable salt or solvate thereof, comprising SEQ ID NO: 1, SEQ ID NO: 2, SEQ ID NO: 3, SEQ ID NO: 13, SEQ ID NO: 14, or SEQ ID NO: 15; and
a therapeutically effective amount of polymyxin B.

34-37. (canceled)

38. A peptide compound having a structure selected from:
(COG1410) acetyl-AS-aib-LRKL-aib-KRLL-amide (SEQ ID NO: 1);
Picolinyl-AS-aib-LRKL-aib-KRLL-amide (SEQ ID NO: 2);
Picolinyl-AS-C-LRKL-aib-KRLL-C-amide (SEQ ID NO: 3), wherein there is a disulfide link between the two cysteines;
Acetyl-LLRK(Aib)LKRL(Aib)SA-CONH2 (SEQ ID NO: 4);
Acetyl-llrk(Aib)lkkl(Aib)sa-amide (SEQ ID NO: 5) (small letters represent D amino acids);
Acetyl-as-aib-lrkl-aib-krll-amide (SEQ ID NO: 6), wherein all the amino acid residues are D-amino acids;
Acetyl-LLRK-Aib-LRKL-Aib-SAS-Aib-LRKL-Aib-KRLL-CONH2 (SEQ ID NO: 7);
Acetyl-LRVRCAS(Aib)LRKL(Aib)KRLL-CONH2 (SEQ ID NO: 8);
Acetyl-LRVRLAS(Aib)LKKL(Aib)KRLL-Amide (SEQ ID NO: 9);
Acetyl-LRVRLAS(Aib)LRKL(Aib)KRLL-Amide (SEQ ID NO: 10);
Acetyl-llrk(aib)lkrl(aib)salrvrl-amide (SEQ ID NO: 11) (small letters represent D amino acids); and
Acetyl-LRVRLASHLRKLRKRLLAS-aib-LRKL-aib-KRLL-CONH2 (SEQ ID NO: 12);
C8-AS-aib-LRKL-aib-KRLL-amide (SEQ ID NO: 13);
Acetyl-K(C8)-AS-aib-LRKL-aib-KRLL-amide (SEQ ID NO: 14);
Acetyl-K(Picolinyl)-AS-aib-LRKL-aib-KRLL-amide (SEQ ID NO: 15);
Acetyl-LRVRLASHLRKLRKRLLR-amide (SEQ ID NO: 16);
Acetyl-LRKLRKRLLLRKLRKRLL-amide (SEQ ID NO: 17);
Acetyl-LRVRLASHLRKLRKRLLRDADDLQKRLAVY-amide (SEQ ID NO: 18); and
Picolinyl-llrk-aib-lkrl-aib-salrvrl-amine (SEQ ID NO: 19), wherein all amino acid residues are D-amino acids,
wherein aib is amino isobutyric acid.

39-51. (canceled)
